SEQUENCE LISTING (26 sequences)

SEQ ID NO: 1 - protein, 355 aa, Homo sapiens
  Wild type SIRT6 amino acid sequence
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 2 - protein, 355 aa, Artificial Sequence
  SIRT6 N308K variant amino acid sequence
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRIKGSIPAGPKQEPCAQHNGSEPASPKRERPTSPAPHRPPKRVKAKAVPS

SEQ ID NO: 3 - protein, 355 aa, Artificial Sequence
  SIRT6 A313S variant amino acid sequence
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPSGPKQEPCAQHNGSEPASPKRERPTSPAPHRPPKRVKAKAVPS

SEQ ID NO: 4 - protein, 355 aa, Homo sapiens
  SIRT6 N308K A313S variant amino acid sequence
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRIKGSIPSGPKQEPCAQHNGSEPASPKRERPTSPAPHRPPKRVKAKAVPS

SEQ ID NO: 5 - DNA, 1068 bp, Homo sapiens
  Wild type SIRT6 nucleic acid sequence
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 6 - DNA, 1068 bp, Artificial Sequence
  SIRT6 N308K variant nucleic acid sequence
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaagggctctatccccgccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 7 - DNA, 1068 bp, Artificial Sequence
  SIRT6 A313S variant nucleic acid sequence
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatcccctccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 8 - DNA, 1068 bp, Homo sapiens
  SIRT6 N308K A313S variant nucleic acid sequence
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaagggctctatcccctccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 9 - DNA, 21 bp, Artificial Sequence
  LINE1 ORF1 Forward-primer
atggcgaaaggcaaacgtaag

SEQ ID NO: 10 - DNA, 21 bp, Artificial Sequence
  LINE1 ORF1 Reverse-primer
attttcggttgtgttggggtg

SEQ ID NO: 11 - DNA, 21 bp, Artificial Sequence
  LINE1 ORF2 Forward-primer
gcaggggttgcaatcctagtc

SEQ ID NO: 12 - DNA, 21 bp, Artificial Sequence
  LINE1 ORF2 Reverse-primer
ctgggtgctcctgtattgggt

SEQ ID NO: 13 - DNA, 21 bp, Artificial Sequence
  N1SMA forward primer
aaaagacagctacgtgggtga

SEQ ID NO: 14 - DNA, 22 bp, Artificial Sequence
  N1SMA reverse primer
gccatgttctatcgggtacttc

SEQ ID NO: 15 - DNA, 21 bp, Artificial Sequence
  COL1A1 forward primer
gtgcgatgacgtgatctgtga

SEQ ID NO: 16 - DNA, 19 bp, Artificial Sequence
  COL1A1 reverse primer
cggtggtttcttggtcggt

SEQ ID NO: 17 - DNA, 23 bp, Artificial Sequence
  TIMP1 forward primer
accaccttataccagcgttatga

SEQ ID NO: 18 - DNA, 21 bp, Artificial Sequence
  TIMP1 reverse primer
ggtgtagacgaaccggatgtc

SEQ ID NO: 19 - DNA, 21 bp, Artificial Sequence
  VIMENTIN forward primer
agtccactgagtaccggagac

SEQ ID NO: 20 - DNA, 21 bp, Artificial Sequence
  VIMENTIN reverse primer
catttcacgcatctggcgttc

SEQ ID NO: 21 - DNA, 23 bp, Artificial Sequence
  MMP2 forward primer
tacaggatcattggctacacacc

SEQ ID NO: 22 - DNA, 19 bp, Artificial Sequence
  MMP2 reverse primer
ggtcacatcgctccagact

SEQ ID NO: 23 - DNA, 17 bp, Artificial Sequence
  GAPDH forward primer
ggtgcgtgcccagttga

SEQ ID NO: 24 - DNA, 19 bp, Artificial Sequence
  GAPDH reverse primer
tactttctccccgcttttt

SEQ ID NO: 25 - DNA, 21 bp, Artificial Sequence
  ActinN2 forward primer
catgtacgttgctatccaggc

SEQ ID NO: 26 - DNA, 21 bp, Artificial Sequence
  ActinN2 reverse primer
ctccttaatgtcacgcacgat

(19) United States
(12) Patent Application Publication
(10) Pub. No.: US 2024/0277868 A1
(43) Pub. Date: Aug. 22, 2024

(54) VARIANTS OF SIRT6 FOR USE IN PREVENTING AND/OR TREATING AGE-RELATED DISEASES

(71) Applicants: UNIVERSITY OF ROCHESTER, Rochester, NY (US); THE TRUSTEES OF COLUMBIA UNIVERSITY IN THE CITY OF NEW YORK, New York, NY (US); ALBERT EINSTEIN COLLEGE OF MEDICINE, Bronx, NY (US)

(72) Inventors: Vera GORBUNOVA, Honeoye Falls, NY (US); Andrei SELUANOV, Honeoye Falls, NY (US); Yousin SUH, New York, NY (US)

(73) Assignees: UNIVERSITY OF ROCHESTER, Rochester, NY (US); THE TRUSTEES OF COLUMBIA UNIVERSITY IN THE CITY OF NEW YORK, New York, NY (US); ALBERT EINSTEIN COLLEGE OF MEDICINE, Bronx, NY (US)

(21) Appl. No.: 18/560,828

(22) PCT Filed: May 13, 2022
(86) PCT No.: PCT/US2022/029213

§ 371 Date: Nov. 14, 2023

Related U.S. Application Data
(60) Provisional application No. 63/188,573, filed on May 14, 2021.

(51) Int. Cl. A61K 48/00; A61K 38/45; A61P 35/00; A61P 39/00; C12N 9/10

(52) U.S. Cl. CPC A61K 48/0016; A61K 38/45; A61P 35/00; A61P 39/00; C12N 9/1029

(57) ABSTRACT

An isolated nucleic acid molecule encoding a variant of sirtuin 6 (SIRT6) having at least 75% identity with sequence SEQ ID NO: 1, the variant having at least one mutation selected from a N308K substitution and a A313S substitution with respect to sequence SEQ ID NO: 1. Also, means for the regulation of ageing, and/or senescence, and/or lifespan in an individual. Further, means for the repair of double strand breaks in a cell. Finally, means for the prevention and/or the treatment of an age-related disease.

Statement regarding federally sponsored research or development. This invention was made with government support under AG056278, AG027237, AG047200 awarded by the National Institutes of Health. The government has certain rights in the invention.

### FIELD OF INVENTION

The present invention relates to lifespan, longevity and age-related diseases. More particularly, the invention relates to variants of sirtuin 6 (SIRT6), and therapeutic uses thereof, for the regulation of ageing and for treating and/or preventing age-related diseases.

### BACKGROUND OF INVENTION

Living to a hundred years-old has a strong genetic component, and is yet extremely rare, with a prevalence in developed nations of at most 1 per 6,000. Genome-wide association studies have so far only identified APOE as a significative biomarker associated with extreme longevity in humans, likely because of a gross lack of statistical power.

However, a great deal of diseases, referred to as age-related diseases, occur often as the individual gets older.

Examples of such age-related diseases include, e.g., cardiovascular diseases, strokes, hypertension, cancer, type 2 diabetes, Parkinson's disease, Alzheimer's disease and other dementia, chronic obstructive pulmonary disease (COPD), osteoarthritis, osteoporosis, cataract, age-related macular degeneration, hearing loss.

It is now believed that age-related diseases are the consequence of a decreased capacity of the body to repair DNA damages and to maintain the integrity of telomeres.

To date, a family of proteins called the sirtuins (SIRTs), has been associated with various mechanisms involved in DNA damage repair, telomeric integrity, senescence, and longevity.

Among the SIRTs proteins, SIRT6, encoded by the SIRT6 gene regulates the expression of telomere reverse transcriptase required for telomere elongation, as well as deacetylate histone 3 lysine 9 (H3K9) and deacetylate histone 3 lysine 56 (H3K56) resulting in maintaining the telomeric integrity. In addition, SIRT6 gene was shown to be recruited to the damaged sites and promote DNA repair through deacetylating the repair proteins such as poly (ADP-ribose) polymer-ase (PARP)-1, Ku70, NBS, and Werner (WRN) helicase. Especially, SIRT6 acts as a transcriptional regulator to suppress gene expression by stabilizing the chromatin structure.

Furthermore, SIRT6 modulates cellular senescence through the deacetylation of a variety of signaling molecules such as FOXO and p53. SIRT6 regulates the RelA subunit of NFκB by modifying the cellular senescence-related gene expression. Finally, SIRT6 also reduces cytotoxicity and therefore decreases the damage that causes premature senescence.

Therefore, SIRT6 may constitute a good target to prevent and/or treat age-related diseases.

There is still a need to provide means to prevent and/or treat age-related diseases. In particular, there is a need to prevent and/or treat senescence-related disorder, in particular premature senescence. There is also a need to improve DNA damage repairs involved in senescence, including premature senescence, and decreased longevity.

### SUMMARY

A first aspect of the invention relates to an isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1.

In some embodiments, the nucleic acid molecule is of sequence selected in the group comprising or consisting of SEQ ID NO: 6, SEQ ID NO: 7 and SEQ ID NO: 8.

Another aspect of the invention pertains to an isolated polypeptide encoded by a nucleic acid molecule as defined herein.

In certain embodiments, wherein the polypeptide is of sequence selected in the group comprising or consisting of SEQ ID NO: 2, SEQ ID NO: 3 and SEQ ID NO: 4.

A further aspect of the invention relates to a vector comprising the isolated nucleic acid molecule as defined herein.

In some embodiments, the vector is a viral vector, in particular an adeno-associated viral vector (AAV), an exosome-associated AAV vector (exo-AAV), an adenoviral vector, a retroviral vector, or a herpes virus vector.

In one aspect, the invention relates to a suspension comprising a vector as defined herein.

The invention also pertains to a cell expressing the polypeptide as defined herein, the cell being preferably transfected with an isolated nucleic acid molecule, or a vector as defined herein.

Another aspect of the invention relates to a pharmaceutical composition comprising (i) an isolated nucleic acid molecule, or an isolated polypeptide, or a vector as defined herein, and (ii) a pharmaceutically acceptable excipient.

The invention relates to the use of an isolated nucleic acid molecule, or an isolated polypeptide, or a vector as defined herein, in the regulation of ageing, and/or senescence, and/or lifespan in an individual, preferably a mammalian individual, more preferably a human individual.

A further aspect of the invention pertains to the use of an isolated nucleic acid molecule, or an isolated polypeptide, or a vector according as defined herein, in the repair of double strand break in a cell, preferably a mammalian cell, more preferably a human cell.

In some aspects, the invention also relates to an isolated nucleic acid molecule, or an isolated polypeptide, or a vector as defined herein, for use in the prevention and/or the treatment of an age-related disease.

In certain embodiments, the individual is a mammalian individual, preferably a human individual.

In some embodiments, the age-related disease is selected in the group comprising or consisting of progeria, Werner syndrome, neurodegenerative disease, Alzheimer's disease, cancer, cardiovascular disease, obesity, type 2 diabetes, hypercholesterolemia, hypertension, ocular disorders, cataracts, glaucoma, osteoporosis, blood clotting disorders, arthritis, hearing loss and stroke.

In another aspect, the invention also relates to a kit comprising (i) an isolated nucleic acid molecule, or an isolated polypeptide, or a vector as defined herein, and (ii) means to administer the isolated nucleic acid molecule, the isolated polypeptide, or the vector.

### Definitions

In the present invention, the following terms have the following meanings:

“About”, when preceding a figure, means plus or less 10% of the value of said figure. It is to be understood that the value to which the term “about” refers is itself also specifically, and preferably, disclosed.

“Comprise” is intended to mean “contain”, “encompass” and “include”. In some embodiments, the term “comprise” also encompasses the term “consist of”.

“Sirtuin 6” also referred to as “SIRT6”, is intended to refer to the polypeptide with the Entrez Gene number 51548, and also non-limitatively relates to the NAD-Dependent Protein Deacetylase Sirtuin-6, Regulatory Protein SIR2 Homolog 6, SIR2-Like Protein 6, SIR2L6, Sirtuin (Silent Mating Type Information Regulation 2, S. Cerevisiae, Homolog) 6, Sirtuin (Silent Mating Type Information Regulation 2 Homolog) 6, Sir2-Related Protein Type 6, Sirtuin Type 6 and EC 2.3.1.286.

“Isolated” refers to a nucleic acid molecule or polypeptide a that is removed from the initial biological context that has allowed to generate this nucleic acid molecule or polypeptide a. In practice the biological context comprises at least a cell, or one or more enzyme(s).

“Nucleic acid”, also referred to as “polynucleotide”, refers to any polyribonucleotide or polydeoxyribonucleotide, which may be unmodified RNA or DNA or modified RNA or DNA. “Nucleic acid” or “polynucleotide” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Nucleic acid” or “polynucleotide” refers to triple-stranded regions comprising RNA or DNA or both RNA and DNA. The term “nucleic acid” or “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” or “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acid residues other than the 20 gene-encoded amino acid residues.

“Age-related disease” refers to the physiological or pathological conditions that correlate with an individual getting older or prematurely older, and which prevalence is above the average prevalence observed within the general population. Non-limitative examples of age-related diseases include progeria, Werner syndrome, neurodegenerative diseases, cardiovascular diseases, strokes, hypertension, cancer, type 2 diabetes, Parkinson's disease, dementia, chronic obstructive pulmonary disease (COPD), osteoarthritis, osteoporosis, cataract, age-related macular degeneration, hearing loss, and the like.

“Suspension” refers to a liquid mixture in which the active principle, such as the nucleic acid molecules, polypeptides, or vectors according to the invention, is/are floating in a liquid medium.

“Treating” or “treatment” or “alleviation” refers to both therapeutic treatment and prophylactic or preventative measures, wherein the object is to prevent or slow down (lessen) the targeted pathologic condition or disorder, in particular an age-related disease. Those in need of treatment include those already with said disorder as well as those prone to develop the disorder or those in whom the disorder is to be prevented. An individual is successfully “treated” for an age-related disease if, after receiving a therapeutic amount of the active principle, in particular the nucleic acid molecules, polypeptides, or vectors according to the present invention, the individual shows observable and/or measurable reduction in or absence of one or more of the symptoms associated with the age-related disease; reduced morbidity and mortality, and improvement in quality of life issues. The above parameters for assessing successful treatment and improvement in the disease are readily measurable by routine procedures familiar to physician or authorized personnel.

“Preventing” refers to keeping from happening, and/or lowering the chance of the onset of, or at least one adverse effect or symptom of, an age-related disease, disorder or condition associated with a deficiency in or absence of an organ, tissue or cell function.

“Therapeutically efficient amount” refers to the level or the amount of the active principle that is aimed at, without causing significant negative or adverse side effects to the target, (1) delaying or preventing the onset of an age-related disease, disorder, or condition; (2) slowing down or stopping the progression, aggravation, or deterioration of one or more symptoms of an age-related disease, disorder, or condition; (3) bringing about ameliorations of the symptoms of an age-related disease, disorder, or condition; (4) reducing the severity or incidence of an age-related disease, disorder, or condition; or (5) curing an age-related disease, disorder, or condition. A therapeutically effective amount may be administered prior to the onset of an age-related disease, disorder, or condition, for a prophylactic or preventive action. Alternatively, or additionally, the therapeutically effective amount may be administered after the onset of an age-related disease, disorder, or condition, for a therapeutic action. In one embodiment, a therapeutically effective amount of the active principle is an amount that is effective in reducing at least one symptom of an age-related disease, disorder or condition.

“Individual” refers to an animal, preferably a mammal, more preferably a human. In one embodiment, the individual is a male. In another embodiment, the individual is a female. In one embodiment, an individual may be a “patient”, i.e. a warm-blooded animal, more preferably a human, who/which is awaiting the receipt of, or is receiving medical care or was/is/will be the object of a medical procedure, or is monitored for the development of cancer. In one embodiment, the individual is an adult (for example a subject above the age of 18). In another embodiment, the individual is a child (for example a subject below the age of 18).

### BRIEF DESCRIPTION OF THE DRAWINGS

FIG. 1 is a scheme showing the screening strategy for variants in genome maintenance (GM) genes in Ashkenazi Jewish (AJ) centenarians.

FIG. 2 is a plot showing the purified SIRT6 proteins turn-over rate. SILAC analysis on HEK293 cells expressing SIRT6 variants.

FIG. 3 is a graph showing the thermostability of purified SIRT6 proteins (WT: wild type SIRT6 polypeptide; Cent: SIRT6 variant polypeptide with N308K and A313S mutations). Data represents two replicates with two technical replicates each using SIRT6 from the Roc and Ichor preps.

FIGS. 4A-4B are a combination of graphs. FIG. 4A shows the fluorescence lifetime t measured from FLT signals by fitting the data to Equation 1 (see Methods). Each of the two different SIRT6 biosensors shows a decreased fluorescence lifetime compared to the GFP-only control, indicating highly significant FRET. Compared with the biosensor with C-terminal GFP, the intra-GFP biosensor shows a greater lifetime change (more FRET, shorter distance R, more structural sensitivity). The centSIRT6 of this biosensor was also tested. It shows a significant increase in lifetime t (1.96+0.02 ns), compared to the wild type SIRT6 (1.75±0.01 ns). Error bars indicate SEM (n=3-5). FIG. 4B shows the FRET efficiency E and the distance R (in Angstrom; Å) were determined from lifetime data (Equations. 2 and 3, Methods), revealing that the distance R, measure for the intra-GFP biosensor, was significantly greater for the centSIRT6 by 3.0±0.4 Å.

FIG. 5A-5B are a combination of graphs. FIG. 5A shows the Michaelis-Menten kinetic parameters using differential concentrations of myristoylated peptide. FIG. 5B shows the Michaelis-Menten kinetic parameters using differential concentrations of NAD+. Reactions were conducted in triplicate. Closed circles: WT; closed squares: Cent.

FIG. 6 is a graph showing the tryptophan fluorescence curves for SIRT6 variants with titrated concentrations of NAD+.

FIG. 7A-7B is a combination of plots showing the deacetylase activity on H3K9ac (FIG. 7A) and H3K18ac (FIG. 7B) residues, with reduced activity in centSIRT6 allele (the deacetylase activity is inversely proportional to the relative abundance of acetylated compounds). Designer histones saturated with the corresponding acetylated histone residue were incubated with purified SIRT6 and 1 mM NAD+ for 1 h prior to resolution by SDS-PAGE gel and staining with acetyl-specific histone antibodies. All reactions were conducted with an n=3; error bars show s.d. Statistics were calculated using Students t-test, two tailed. Asterisk indicate p<0.05.

FIG. 8A-8B is a combination of plots showing the deacetylation kinetics on H3K9ac (FIG. 8A) and H3K18ac (FIG. 8B) residues of SIRT6 variants. Designer histones were incubated with purified SIRT6 and 5 mM NAD+, then resolved by SDS-PAGE and analyzed by immunoblotting with acetyl-specific histone antibodies. All reactions were conducted with an n=3; error bars show s.d. Statistics were calculated using Students t-test, two tailed. Asterisk indicate p<0.05.

FIG. 9A-9B is a combination of plots showing the in vitro deacetylation rates on H3K9ac (FIG. 9A) and H3K18ac (FIG. 8B) residues of purified SIRT6 proteins with histones purified from HeLa cells.

FIG. 10 is a plot showing the quantitative mass spec of histone H3 peptide purified from human cells expressing different SIRT6 alleles, which did not reveal a difference in acetylation levels. Relative fraction represents the portion of the peptide encompassing H3K9-17 compared to the summed total of all of the peptide quantitation values for the same region. The average and standard deviation of three different preps is plotted.

FIG. 11 is a photograph showing the whole cell histone H3 acetylation levels in cumate-inducible SIRT6 human fibroblasts, assessed by Western blot. Cu, cumate; PQ, paraquat. Cumate dosage required for equivalent SIRT6 protein abundance was determined by Western blot, and administered accordingly to respective cell lines.

FIG. 12 is a plot showing the self-ribosylation of SIRT6 using radiolabeled NAD+. Recombinant SIRT6 was incubated with P32-labeled NAD+ and then run on an SDS-PAGE. Signal was measured using a PhosphoImager. All experiments were repeated at least three times; error bars show s.d. Significance was determined by Student's t-test, two tailed. Asterisk indicate p<0.05. Asterisks indicate significance over HPRT control.

FIG. 13 is a plot showing the self-ribosylation of SIRT6 with titration of NAD+. mADPr-specific antibody was used to detect ribosylated wild type SIRT6 (closed circles) and centSIRT6 (closed squares) proteins. Plot was fit the Michaelis-Menten equation with Kaleidagraph software. Km NAD was 142+29 for centSIRT6 and 106+45 for wild type SIRT6 and maximal signal was ˜2× greater for centSIRT6 compared to the wild type SIRT6.

FIG. 14 is a graph showing the activation of PARPI by SIRT6 variants. SIRT6 protein was incubated with human PARP1 protein and then analyzed by immunoblotting with poly-ADPr antibody. Poly-ADPr activity of PARP1 results in a wide range of product size. Activity was assessed by quantifying poly-ADPr signal in whole lanes for each sample. All experiments were repeated at least three times; error bars show s.d. Significance was determined by Student's t-test, two tailed. Asterisk indicate p<0.05. Lower asterisks indicate significance over HPRT control. Upper asterisks indicated significance over wild type SIRT6.

FIG. 15 is a graph showing the qRT-PCR analysis of LINE1 expression in cumate-inducible SIRT6 fibroblasts. Primers assessed both 5′ (ORF1; closed bars) and 3′ (ORF2; dashed bars) LINE1 sequences from the LIMdA1 family of active LINE1 retrotransposons. Assessment of both regions was conducted to mitigate contributions from partial insertion sequences in coding genes. All experiments were repeated at least three times. Error bars represent s.d. Significance was determined by Student's t-test, two tailed, unless otherwise stated. Asterisk indicate p<0.05.

FIG. 16A-16B is a combination of graphs showing the stimulation of NHEJ (FIG. 16A) and HR (FIG. 16B) by SIRT6 variants. Reporter cell lines were co-transfected with SIRT6-expressing plasmid, I-Scel plasmid, and DsRed transfection control. After 72 hr recovery, reactivation of the GFP reporter was measured by flow cytometry. Stimulation of NHEJ or HR was calculated as radio of GFP+/DsRed+ positive cells.

FIG. 17 is a graph showing the basal γH2AX foci in cumate-inducible SIRT6 fibroblasts under uninduced (white bars) or induced (black bars) conditions. Foci were scored in at least 80 cells per condition.

FIG. 18 is a plot showing the DNA repair kinetics in cumate-inducible SIRT6 fibroblasts. Cells were grown on slides and irradiated with 2 Gy gamma radiation, followed by immunostaining for γH2AX. Irradiation was conducted when the cells were at 75% confluency on slides. Cells were fixed and foci scored at t=0.5 h, 2 h, 4 h, 6 h, and 24 h post-irradiation. Foci were scored in at least 80 cells per genotype per timepoint. Asterisk indicates significant difference from the wild type SIRT6 (p<0.05).

FIG. 19A-19B is a combination of plots showing the oxidative stress resistance. Cumate-inducible SIRT6 fibroblasts were induced for SIRT6 expression and exposed to paraquat for 24 hours. Resistance was determined by apoptosis staining 48 hours after exposure.

FIG. 20A-20C is a combination of graphs. FIG. 20A is showing the number of adherent cells after transfection with SIRT6 variants. Cells were transfected with SIRT6 plasmids encoding different SIRT6 alleles and cell numbers were counted after 72 hours. HCA2 are normal human foreskin fibroblasts. a: Control (CT); b: WT; c: N308K SIRT6 variant; d: A313S SIRT6 variant; e: Cent (N308K A313S variant). Asterisk indicates significant difference from the wild type SIRT6 (p<0.05). FIG. 20B-20C are showing the apoptosis staining of cancer cell lines HT1080 (FIG. 20B) and HeLa (FIG. 20C) 48 hours after transfection. Cells were stained with Annexin V/PI and analyzed by flow cytometry. Significance was determined by two-way ANOVA. All experiments were repeated at least three times. Error bars represent s.d. Significance was determined by Student's t-test, two tailed, unless otherwise stated. Asterisk indicate p<0.05.

FIG. 21 is a photograph showing the immunoprecipitation (IP) experiments on lysates from cumate-induced fibroblasts expressing wild type (WT) or centSIRT6 (Cent) alleles with antibodies to SIRT6, LMNA, and mADPr. SIRT6 expression was induced 48 hours prior to IP. The IP experiments were repeated three times. One representative set of IPs is shown.

FIG. 22 is a graph showing SIRT6 IP followed by Western blot with antibodies to LMNA, in which CentSIRT6 (Cent) shows stronger interaction with LMNA compared to the wild type SIRT6 (WT). Quantification of the IP experiment shown in FIG. 21.

FIG. 23 is a graph showing LMNA IP from cumate-inducible SIRT6 fibroblasts followed by Western blot with antibodies to SIRT6, in which LMNA shows enhanced interaction with centSIRT6 (Cent) compared to the wild type (WT). Quantification of the IP experiment shown in FIG. 21.

FIG. 24 is a graph showing the quantification of the IP experiment shown in FIG. 21. SIRT6 IP from cumate-inducible SIRT6 fibroblasts followed by Western blot with antibody to mADPr residues shows that centSIRT6 displays enhanced mADPr.

FIG. 25 is a graph showing the quantification of the IP experiment shown in FIG. 21. IP with mADPr antibody using extract from cumate-induced SIRT6 fibroblasts, followed by Western blot with antibodies to LMNA, shows that LMNA displays enhanced mADPr signal in cells expressing centSIRT6 (Cent), as compared to wild type (WT).

FIG. 26 is a scheme showing protein-protein interactions profiles of SIRT6 and LMNA. SIRT6 and LMNA are colored dark grey and featured as central points in two opposing nodes of interactions. A third node (upper middle) shows interaction partners that are shared by SIRT6 and LMNA. Proteins whose interactions are enhanced by the centSIRT6 allele are colored light grey. Proteins that interacted equally with wild type and centSIRT6 alleles are uncolored. H15 is a special case because it showed increased interaction with the centSIRT6 and decreased interaction with LMNA in the presence of the centSIRT6 allele. Proteins known to be ribosylated in previous reports are shown as hexagons.

FIG. 27A-27B is a combination of histograms. FIG. 27A shows SIRT6 expression in cumate-inducible telomerase-immortalized HCA2 human fibroblast cell lines. SIRT6 alleles were integrated in the genome of SIRT6 knockout HCA2 cells using PiggyBac Transposon Vector System. Different dosages of cumate and resulting SIRT6 abundance were used to determine the dose needed to achieve equivalent SIRT6 expression for each cell line. Cells were normalized by count and total protein. Subsequent experiments utilizing cumate-inducible SIRT6 fibroblasts were controlled for SIRT6 abundance using these data. For each cell line, the box indicates the corresponding cumate dosage for equivalent expression (WT=60 μg/ml, N308K=30 μg/ml, A313S=30 μg/ml, and Cent=7.5 μg/ml). These concentrations were used in experiments with these cells. FIG. 27B shows qRT-PCR expression analysis of SIRT6 using standardized dosages of cumate (dosage corresponds to boxes in FIG. 27A).

FIG. 28 is a histogram showing Self-ribosylation of SIRT6 using biotin-labeled NAD+. Recombinant SIRT6 was incubated with NAD+ conjugated with a biotin residue and then run on an SDS-PAGE. Each allele was assessed relative to its 0 hr time point and normalized to SIRT6 total protein loading controls.

FIG. 29A-29F is a combination of photographs, graphs and histograms showing the analysis of CRISPR human MSC cell lines. FIGS. 29A and 29B show DNA double strand repair efficiency in wild type and centSIRT6 (“Cent #1” and “Cent #2”) hMSCs. DSB repair reporter constructs were integrated into hMSCs. After 72 hr recovery, reactivation of the GFP reporter was measured by flow cytometry. Stimulation of NHEJ or HR was calculated as radio of GFP+/DsRed+ positive cells. FIG. 29C shows cell viability in MMS-treated wild type and centSIRT6 (“Cent”) hMSCs. hMSCs were treated with MMS for 48 hours and cell viability was evaluated by MTS assay. Data were normalized to the control group (0 mM). n=6. FIG. 29D shows immunofluorescence staining of 53BP1 in wild type and centSIRT6 (“Cent”) hMSCs under MMS treatment. Numbers of 53BP1 foci in the nuclei of wild type and centSIRT6 hMSCs with or without MMS (0.25 mM) treatment were quantified. >600 nuclei from 10 images were scored. FIG. 29E shows qRT-PCR analysis of SIRT6 expression in wild type and centSIRT6 (“Cent #1” and “Cent #2”) hMSCs (P2). Data normalized to Actin and were presented as mean±SEM, NS, not significant. FIG. 29F shows this quantification. Error bars represent s.d. Significance was determined by Student's t-test, two tailed. Asterisk indicate p<0.05.

FIG. 30A-30C is a combination of photograph and histograms illustrating overexpression of SIRT6 (wild type, SIRT6 N308K or centSIRT6 (“Cent”)) in human Werner Syndrome immortalized fibroblasts. FIG. 30A shows a proliferation assay of the same cells, using Hoechst fluorescence staining at 24 h, 48 h and 72 h. Data are showed as mean+SEM. FIG. 30B shows a telomere length assay showing significant difference in all the transfected cells when compared to not transfected control cells (*=versus control). FIG. 30C shows that telomerase activity assay identified a significant decrease in centSIRT6 group (“Cent”) when compared to not transfected control cells.

FIG. 31A-31B is a combination of photograph and histogram showing that SIRT6 overexpression (wild type, SIRT6 N308K or centSIRT6 (“Cent”)) induces cell death in hepatocellular carcinoma cell lines, after 12 days LV transfection and 3 days after hygromycin selection. FIG. 31A shows that in human hepatocellular carcinoma (HCC) cells, SIRT6 WT/SIRT6 N308K/centSIRT6 (“Cent”) overexpression was lethal, with a larger effect observed for SIRT6 N308K/centSIRT6 (“Cent”). FIG. 31B represents the quantification of cell viability illustrated in FIG. 31A.

FIG. 32 is a graph showing that SIRT6 overexpression (wild type, SIRT6 N308K or centSIRT6 (“Cent”)) increase differentiation of 3T3-L1 pre-adipocytes (quantification of immunofluorescence).

FIG. 33A-33E is a combination of photograph and histograms showing the effect of SIRT6 overexpression (wild type, SIRT6 N308K and centSIRT6 (“Cent”)) in hepatic stellate cells (LX2). FIG. 33A shows the viability of LX2 cells after 24 h of transfection with SIRT6 (WT or mutants). FIG. 33B-33E shows gene expression (mRNA) in LX2 cells overexpressing or not SIRT6 (WT or mutants) after 24 h TGF-β 20 ng treatment for Vimentin (FIG. 33B), TIMP1 (FIG. 33C), COLIA1 (FIG. 33D) and aSMA (FIG. 33E) (N=4; *=p<0.05 versus respective control).

FIG. 34A-34C is a combination of histograms showing the effect of SIRT6 overexpression (wild type, SIRT6 N308K and centSIRT6 (“Cent”)) on spheroids made from Immortalized Human Hepatocytes (IHH) and human hepatic stellate cells (LX2). Spheroids are formed from IHH cells and with 5% the total cell mass of LX2. FIG. 34A shows the quantification of collagen I in IHH-LX2 spheroids overexpressing or not centSIRT6 (“Cent”) (quantification of immunofluorescence). FIG. 34B-34C shows IHH-LX2 spheroids gene expression relative to GAPDH for aSMA, COL1A1, TIMP1, VIMENTIN and MMP2, either in absence of Free Faty Acids (FFA) (FIG. 34B) or in presence of FFA (FIG. 34C) (* p<0.05; ** p<0.01; *** p<0.001 versus respective control).

### DETAILED DESCRIPTION

The inventors used herein an alternative, candidate functional association approach to directly identify rare longevity gene variants enriched in the genome of a cohort of Ashkenazi Jewish (AJ) centenarians.

Based on previous evidences of DNA damage as a driver of ageing, the inventors performed targeted sequencing of 301 genes involved in Genome Maintenance (GM), a major longevity assurance system, in 496 AJ centenarians and 572 controls.

Among the ten GM genes containing nominally significant missense genetic variants enriched in the centenarian genome, was SIRT6, a deacylase and mono-ADP ribosyl transferase (mADPr) enzyme-6, involved in both DNA double-strand breaks (DSB) repair and longevity in model organisms.

The inventors selected two genetically linked variants in the same allele (N308K and A313S), for further functional analysis. Characterization of this SIRT6 centenarian allele (centSIRT6) demonstrated it to be a stronger suppressor of LINE1 retrotransposons, confer enhanced stimulation of DNA DSB repair, and more robust cancer cell killing compared to the wild type allele.

Surprisingly, centSIRT6 displayed weaker deacetylase activity compared to the wild type. Conversely, its mADPr activity was strongly enhanced. FRET-based analysis demonstrated the centSIRT6 had a more open conformation. centSIRT6 displayed a stronger interaction with Lamin A/C (LMNA), which correlated with enhanced ribosylation of LMNA.

This invention relates to an isolated nucleic acid molecule encoding a variant of sirtuin 6 (SIRT6) having at least 75% identity with sequence SEQ ID NO: 1, the variant having at least one mutation selected in the group comprising or consisting of a N308K substitution and an A313S substitution with respect to sequence SEQ ID NO: 1.

As used herein the expression “at least 75% identity” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% identity.

The level of identity of 2 polypeptides may be performed by using any one of the known algorithms available from the state of the art. Illustratively, the amino acid identity percentage may be determined using the CLUSTAL W software (version 1.83), the parameters being set as follows:
for slow/accurate alignments: (1) Gap Open Penalty: 10.00; (2) Gap Extension Penalty:0.1; (3) Protein weight matrix: BLOSUM;
for fast/approximate alignments: (4) Gap penalty: 3; (5) K-tuple (word) size: 1; (6) No. of top diagonals: 5; (7) Window size: 5; (8) Scoring Method: PERCENT.

In certain embodiments, the isolated nucleic acid molecule encodes a variant of SIRT6 having at least 80%, preferably at least 85%, more preferably at least 90%, even more preferably at least 95%, most preferably at least 96%, 97%, 98% or 99% identity with sequence SEQ ID NO: 1, the variant having at least one mutation selected in the group comprising or consisting of a N308K substitution and an A313S substitution with respect to sequence SEQ ID NO: 1.

Within the scope of the invention the sequence SEQ ID NO: 1 refers to the 361 amino acid residues sequence of wild type SIRT6 polypeptide. In practice, substitutions N308K and A313S refer to the mutations of the codon encoding the naturally occurring Asn (N) amino acid residue at position 308 in the SIRT6 polypeptide, and the Ser (S) amino acid residue at position 313 in the SIRT6 polypeptide, respectively.

Within the scope of the invention the sequence SEQ ID NO: 5 refers to the 1,068 nucleotides (bp) sequence of wild type SIRT6 polypeptide.

In some embodiments, the naturally occurring Asn (N) amino acid residue at position 308 in the SIRT6 polypeptide is encoded by codon “aac” at positions 922 to 924 of SEQ ID NO: 5. In some embodiments, the naturally occurring Ser (S) amino acid residue at position 313 in the SIRT6 polypeptide is encoded by codon “gcc” at positions 937 to 939 of SEQ ID NO: 5.

In certain embodiments, the N308K substitution is represented by a mutation of codon “aac” at positions 922 to 924 of SEQ ID NO: 5 into codon “aag” or codon “aaa”, preferably into codon “aag”. In other words, the N308K substitution is represented by a mutation of nucleotide “c” at positions 924 of SEQ ID NO: 5 into nucleotide “g” or nucleotide “a”, preferably into nucleotide “g”.

In certain embodiments, the A313S substitution is represented by a mutation of codon “gcc” at positions 937 to 939 of SEQ ID NO: 5 into a codon selected in a group consisting of codons “tcc”, “tct”, “tca” and “tcg”, preferably codon “tcc”. In other words, the A313S substitution is represented by one or two mutation(s) selected in a group consisting of a mutation of nucleotide “g” at positions 937 of SEQ ID NO: 5 into nucleotide “t”; a mutation of nucleotide “g” at positions 937 of SEQ ID NO: 5 into nucleotide “t” and of nucleotide “c” at positions 939 of SEQ ID NO: 5 into nucleotide “t”; a mutation of nucleotide “g” at positions 937 of SEQ ID NO: 5 into nucleotide “t” and of nucleotide “c” at positions 939 of SEQ ID NO: 5 into nucleotide “a”; and a mutation of nucleotide “g” at positions 937 of SEQ ID NO: 5 into nucleotide “t” and of nucleotide “c” at positions 939 of SEQ ID NO: 5 into nucleotide “g”.

In some embodiments, the nucleic acid molecule is of sequence selected in the group comprising or consisting of SEQ ID NO: 6, SEQ ID NO: 7 and SEQ ID NO: 8.

As used herein, sequence SEQ ID NO: 6 refers to the nucleic acid sequence of the variant of SIRT6 with N308K substitution, in particular, with mutation of codon “aac” at positions 922 to 924 of SEQ ID NO: 5 into codon “aag”.

As used herein, sequence SEQ ID NO: 7 refers to the nucleic acid sequence of the variant of SIRT6 with A313S substitution, in particular, with mutation of codon “gcc” at positions 922 to 924 of SEQ ID NO: 5 into codon “tcc”.

As used herein, sequence SEQ ID NO: 8 refers to the nucleic acid sequence of the variant of SIRT6 with N308K and A313S substitutions, in particular, with mutation of codon “aac” at positions 922 to 924 of SEQ ID NO: 5 into codon “aag” and with mutation of codon “gcc” at positions 922 to 924 of SEQ ID NO: 5 into codon “tcc”.

In some embodiments, the variant of SIRT6 encoded by the isolated nucleic acid molecule as defined herein has a deacylase and/or mono-ADP ribosyl transferase (mADPr) activity.

In practice, the deacylase activity and mono-ADP ribosyl transferase (mADPr) activity may be assayed accordingly to any suitable from the state in the art, or a method adapted therefrom. Illustratively, deacylase activity may be assayed by contacting in vitro the variant of SIRT6 with histones, in the presence of NAD+, MgCl₂, DTT and performing a Western blot analysis using anti-H3K9ac and anti-H3K18ac antibodies. Illustratively, mono-ADP ribosyl transferase (mADPr) activity may be assayed by contacting in vitro the variant of SIRT6 with PARP1, in the presence of ZnCl₂, MgCl₂, NAD+, DTT, salmon sperm DNA and performing a Western blot analysis using anti-PADPR antibodies.

In certain embodiments, the variant of SIRT6 has at most about 90%, preferably at most about 50%, more preferably at most about 25% deacylase activity as compared to wild type SIRT6 (of sequence SEQ ID NO: 1). Within the scope of the invention, the expression “at most about 90%” encompasses about 90%, 85%, 80%, 75%, 70%, 65%, 60%, 55%, 50%, 45%, 40%, 35%, 30%, 25%, 20%, 15%, 10%, 5%, 1% or less.

In some embodiments, the variant of SIRT6 has at most least 100%, preferably at least about 200%, more preferably at least about 300% mono-ADP ribosyl transferase (mADPr) activity as compared to wild type SIRT6 (of sequence SEQ ID NO: 1). Within the scope of the invention, the expression “at least about 100%” encompasses about 100%, 120%, 140%, 160%, 180%, 200%, 220%, 240%, 260%, 280%, 300%, 350%, 400%, 450%, 500%, 550%, 600%, 650%, 700%, 750% or more.

Another aspect of the invention relates to an isolated polypeptide encoded by a nucleic acid molecule according to the instant invention.

This invention relates to an isolated polypeptide being a variant of SIRT6 having at least 75% identity with sequence SEQ ID NO: 1, the variant having at least one mutation selected in the group comprising or consisting of a substitution N308K and a substitution A313S with respect to sequence SEQ ID NO: 1.

As used herein the expression “at least 75% identity” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% identity.

In certain embodiments, the isolated polypeptide being a variant of SIRT6 has at least 80%, preferably at least 85%, more preferably at least 90%, even more preferably at least 95%, most preferably at least 96%, 97%, 98% or 99% identity with sequence SEQ ID NO: 1, the variant having at least one mutation selected in the group comprising or consisting of a substitution N308K and a substitution A313S with respect to sequence SEQ ID NO: 1.

In some embodiments, the polypeptide is of sequence selected in the group comprising or consisting of SEQ ID NO: 2, SEQ ID NO: 3 and SEQ ID NO: 4.

As used herein, sequence SEQ ID NO: 2 refers to the amino acid sequence of the variant of SIRT6 with N308K substitution. In some embodiments, the polypeptide of sequence SEQ ID NO: 2 is encoded by a nucleic acid molecule of sequence SEQ ID NO: 6.

As used herein, sequence SEQ ID NO: 3 refers to the amino acid sequence of the variant of SIRT6 with A313S substitution. In some embodiments, the polypeptide of sequence SEQ ID NO: 3 is encoded by a nucleic acid molecule of sequence SEQ ID NO: 7.

As used herein, sequence SEQ ID NO: 4 refers to the amino acid sequence of the variant of SIRT6 with N308K and A313S substitutions. In some embodiments, the polypeptide of sequence SEQ ID NO: 4 is encoded by a nucleic acid molecule of sequence SEQ ID NO: 8.

In certain embodiments, the polypeptide is a recombinant polypeptide. As used herein, the term “recombinant polypeptide” refers to a polypeptide encoded by an engineered nucleic acid and synthesized upon transformation of said engineered nucleic acid into a microorganism or transfection in an eukaryotic cell for synthesis purposes.

A further aspect of the invention relates to a vector comprising the isolated nucleic acid molecule according to the invention.

In some embodiments, the vector in a minicircle nucleic acid, a plasmid, a cosmid or a bacterial artificial chromosome.

As used herein, the term “minicircle nucleic acid” encompasses non-viral vectors that merely comprise a gene expression cassette and are free of viral and/or bacterial backbone DNA elements from standard plasmids.

As used herein, the term “plasmid” is intended to refer to a small extra-genomic DNA molecule, most commonly found as circular double stranded DNA molecules that may be used as a cloning vector in molecular biology, to make and/or modify copies of DNA fragments up to about 15 kb (i.e., 15,000 base pairs). Plasmids may also be used as expression vectors to produce large amounts of proteins of interest encoded by a nucleic acid sequence found in the plasmid downstream of a promoter sequence.

A used herein, the term “cosmid” refers to a hybrid plasmid that contains cos sequences from Lambda phage, allowing packaging of the cosmid into a phage head and subsequent infection of bacterial cell wherein the cosmid is cyclized and can replicate as a plasmid. Cosmids are typically used as cloning vector for DNA fragments ranging in size from about 32 to 52 kb.

As used herein, the term “bacterial artificial chromosome” or “BAC” refers to extra-genomic nucleic acid molecule based on a functional fertility plasmid that allows the even partition of said extra-genomic DNA molecules after division of the bacterial cell. BACs are typically used as cloning vector for DNA fragment ranging in size from about 150 to 350 kb.

In practice, the vector comprising the nucleic acid molecule encoding a variant of SIRT6 may be in the form of a plasmid, in particular resulting from the cloning of a nucleic acid of interest into a nucleic acid vector. In some embodiments, non-limitative suitable nucleic acid vectors are pBluescript vectors, pET vectors, pETduet vectors, pGBM vectors, pBAD vectors, pUC vectors. In one embodiment, the plasmid is a low copy plasmid. In one embodiment, the plasmid is a high copy plasmid.

In some embodiments, the vector is a viral vector. In some embodiments, the viral vector is selected in a group comprising or consisting of an adenovirus; an adeno-associated virus (AAV); an exosome-associated AAV (exo-AAV); an alphavirus; a herpesvirus; a retrovirus, such as, e.g., a lentivirus or a non-integrative lentivirus; vaccinia virus; a baculovirus; or virus like particles such as, e.g., particles derived from Hepatitis B virus, Parvoviridae, Retroviridae, Flaviviridae, Paramyxoviridae or bacteriophages.

In certain embodiments, the vector is a viral vector, in particular an adeno-associated viral vector (AAV), an exosome-associated AAV vector (exo-AAV), an adenoviral vector, a retroviral vector, or a herpes virus vector.

In some embodiments, the adeno-associated viral vector (AAV) is AAV serotype 2 or AAV serotype 5.

In certain embodiments, the vector, in particular the viral vector, is an exo-AAV vector. As used herein, exo-AAV refers to a vector wherein an adeno-associated virus (AAV) vector, or parts thereof, is associated with an extracellular vesicle (also referred to as exosome), wherein the AAV vector is partially fused, embedded or internalized in the extracellular vesicle. The extracellular vesicle may express specific proteins or markers, for example for targeting purposes.

In certain embodiments, the vector, in particular the viral vector, does not cross the blood-brain barrier. In some alternative embodiments, the vector, in particular viral vector crosses the blood-brain barrier. In practice, the choice of the viral vector may depend of the organ or tissue that is targeted. For example, for brain cancer, neurodegenerative disease, Alzheimer's disease and progeria, a vector that crosses the blood-brain barrier may be preferably selected. On the contrary for obesity, cardiovascular diseases, type 2 diabetes, hypercholesterolemia, ocular diseases, a vector that does not cross the blood-brain barrier may be preferably selected.

In practice, assessing whether a vector, in particular a viral vector, crosses the blood/brain barrier may be performed by any suitable method acknowledged from the state of the art, or a method adapted therefrom. Illustratively, this assessment may be performed by the means of one of the two gold-standard experimental measures of blood/brain barrier permeability, namely, (1) logBB, which is intended to measure the concentration of a compound in the brain divided by concentration in the blood; and (2) logPS, which measures the permeability surface-area product.

In some embodiments, the vector, in particular the viral vector, comprises a promoter sequence suitable for gene expression in mammalian individuals, preferably in human individuals.

Non-limitative examples of promoter sequence suitable for gene expression in mammalian individuals, preferably in human individuals, include CMV (human cytomegalovirus) promoter, EF1a (human elongation factor 1 alpha) promoter, SV40 (Simian vacuolating virus 40) promoter, PGK1 (phosphoglycerate kinase) promoter, Ubc (human ubiquitin C) promoter, and the like.

In certain embodiments, the promoter sequence is preferably the CMV promoter.

In some embodiments, the vector, in particular the viral vector, further comprises a nucleic acid sequence that facilitates the nuclear localization of the polypeptide encoded by the nucleic acid molecule according to the invention into a target recipient cell. In practice, these nuclear localization signal (NLS) have been abundantly discussed in the state of the art.

In certain embodiments, the vector, in particular the viral vector, may comprise a S/MAR (for scaffold/matrix attachment region) nucleic acid sequence. As used herein, the S/MAR nucleic acid sequence, also referred to SAR (for scaffold-attachment region) or MAR (for matrix-associated region), is intended to refer to a nucleic acid sequence that is naturally found in the DNA of eukaryotic chromosomes and that promotes attachment to the nuclear matrix. In some embodiments, the S/MAR nucleic acid sequence may serve as an origin of replication. In practice, vectors comprising a S/MAR nucleic acid sequence may behave as extra-chromosome in a transfected cell and be advantageously transmitted to its progeny. Illustratively, suitable S/MAR nucleic acid sequence may be determined as described in Narwade et al. (Nucleic Acids Research, 2019; 47(14):7247-7261).

In one aspect, the invention relates to a suspension comprising a vector according to the invention.

In some embodiments, the suspension further comprises a fluid including one or more ingredients selected from the group consisting of water, saline, phosphate buffered saline, dextrose, glycerol, ethanol and a combination thereof.

In certain embodiment, the suspension is formulated for intravenous infusion. In practice, the suspension formulated for intravenous infusion may comprise saline (e.g., 0.9% NaCl); lactated Ringers; 5% dextrose; a colloid, such as, e.g., albumin; the like; and any combination thereof.

In one aspect, the invention pertains to a cell expressing the polypeptide, the cell being preferably transfected with an isolated nucleic acid molecule, or a vector according to the instant invention.

In certain embodiments, the cell is a eukaryote cell, preferably an animal cells, more preferably a mammalian cell. As used herein “mammalian cell” includes non-human mammalian cells and human cells. In some embodiments, the cell is a human cell.

In some embodiments, the cell is selected in the group comprising or consisting of nerve cells, bone cells, breast cells, red blood cells, white blood cells, cartilage cells, epithelial cells, endothelial cells, skin cells, muscle cells, bladder cells, kidney cells, liver cells, prostate cells, cervix cells, ovarian cells, pulmonary cells, retinal cells, conjunctival cells, corneal cells, fat cells, and the like.

It is to be understood that the cell according to the invention has been transfected with an isolated nucleic acid molecule according to the invention, or transduced with an isolated nucleic acid molecule according to the invention, or contacted with the vector or the suspension containing the nucleic acid molecule according to the invention. Therefore, the cell contains the nucleic acid molecules either integrated or not in its genome. In practice, because the vector is an expression system, the nucleic acid molecule encoding the variant of SIRT6 is present within the cell in a form allowing its expression and its final location, i.e., the cell nucleus and cytoplasm.

Another aspect of the invention relates to a pharmaceutical composition comprising (i) an isolated nucleic acid molecule, or an isolated polypeptide, or a vector according to the instant invention, and (ii) a pharmaceutically acceptable excipient.

In some embodiments, a suitable pharmaceutically acceptable carrier according to the invention includes any and all conventional solvents, dispersion media, fillers, solid carriers, aqueous solutions, coatings, antibacterial and antifungal agents, isotonic and absorption delaying agents, and the like. In certain embodiments, suitable pharmaceutically acceptable carriers may include, water, saline, phosphate buffered saline, dextrose, glycerol, ethanol and a mixture thereof. In some embodiments, pharmaceutically acceptable carriers may further comprise minor amounts of auxiliary substances such as wetting or emulsifying agents, preservatives or buffers, which enhance the shelf life or effectiveness of the cells. The preparation and use of pharmaceutically acceptable carriers are well known in the art.

One aspect of the invention relates to the use of an isolated nucleic acid molecule, or an isolated polypeptide, or a vector according to the instant invention, in the regulation of ageing, and/or senescence, and/or lifespan in an individual, preferably a mammalian individual, more preferably a human individual.

As used herein, the term “regulating the senescence” is intended to refer to the process of decreasing, reducing or stopping the senescence mechanism, in particular premature senescence.

As used herein, the term “regulating the lifespan” is intended to refer to the process of increasing or improving the lifespan, in other words increasing or improving the duration of the life time course.

The invention further relates to a method for the regulation of ageing, and/or senescence, and/or lifespan in an individual, preferably a mammalian individual, more preferably a human individual, in need thereof, comprising the administration of a therapeutically efficient amount of an isolated nucleic acid molecule, or an polypeptide isolated, or a vector according to the instant invention.

A further aspect of the invention pertains to the use of an isolated nucleic acid molecule, or an isolated polypeptide, or a vector according to the instant invention, in the repair of double strand breaks in a cell, preferably a mammalian cell, more preferably a human cell.

The invention relates also to a method for the repair of double strand breaks in a cell, preferably a mammalian cell, more preferably a human cell, comprising the administration of a therapeutically efficient amount of an isolated nucleic acid molecule, or an isolated polypeptide, or a vector according to the instant invention.

As used herein, “repair of double strand breaks” refers to the process allowing to recover DNA integrity by repair DNA damages consisting in the occurrence of a break in both strands of the double strand DNA nucleic acid. In practice, the repair of double strand breaks involves repair mechanisms such as non-homologous end joining (NHEJ) and homologous repair (HR).

In practice, efficiency of double strand breaks repair may be assessed by any suitable method from the state of the art, or a method adapted therefrom. Illustratively, double strand break repairs efficiency may be assessed by the methods described in Seluanov et al. (J Vis Exp, 2010; doi: 10.3791/2002).

Within the scope of the invention, the nucleic acid molecule, the polypeptide, the vector, the suspension or the pharmaceutical composition according to the invention are intended to increase, improve or ameliorate the efficiency of the DSB repair in the target cell, as compared to the efficiency of the DSB repair in the absence of any treatment, as a reference level.

As used herein, the term “increase, improve or ameliorate the efficiency of the DSB repair” encompasses at least 1.2-fold increase as compared to a reference level. Within the scope of the invention, the term “at least 1.2-fold” encompasses, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.5, 5.0, 5.5, 6.0, 6.5, 7.0, 7.5, 8.0, 8.5, 9.0, 9.5, 10, 15, 20, 25, 30, 35, 40, 45, 50, 60, 70, 80, 90, 100, 200, 300, 400, 500, 600, 700, 800, 900, 1,000-fold, or more.

In another aspect, the invention relates to an isolated nucleic acid molecule, or an isolated polypeptide, or a vector according to the instant invention, for use in the prevention and/or the treatment of an age-related disease.

The invention also relates to the to the use of an isolated nucleic acid molecule, or an isolated polypeptide, or a vector according to the instant invention, for the preparation or the manufacture of a medicament for the prevention and/or the treatment of an age-related disease.

In some further aspect, the invention pertains to a method for the prevention and/or the treatment of an age-related disease in an individual in need thereof, comprising the administration of a therapeutically efficient amount of an isolated nucleic acid molecule, or an isolated polypeptide, or a vector according to the instant invention.

In certain embodiments, the individual in need thereof is a mammalian individual, preferably a human individual.

In some embodiments, the age-related disease is selected in the group comprising or consisting of progeria, Werner syndrome, neurodegenerative disease, Alzheimer's disease, cancer, cardiovascular disease, obesity, type 2 diabetes, hypercholesterolemia, hypertension, ocular disorders, cataracts, glaucoma, osteoporosis, blood clotting disorders, arthritis, hearing loss and stroke.

In one embodiment, the age-related disease is progeria or Werner syndrome.

In some embodiments, the age-related disease is cancer. In certain embodiments, the cancer is a blood cancer or a solid cancer.

As used herein, the term “blood cancer”, also referred to as “hematologic cancer”, encompasses any cancer involving uncontrolled proliferation of blood cells, in particular white blood cells. Blood cancers includes leukemia, lymphoma (Hodgkin and non-Hodgkin lymphomas) and myeloma.

In certain embodiments, the cancer is a blood cancer. In some embodiments, said cancer is a blood cancer selected in the group consisting of Hodgkin's disease, immunoblastic lymphadenopathy, lymphoma, chronic lymphocytic leukemia, acute leukemia, and the like.

As used herein, the term “solid cancer” encompasses any cancer (also referred to as malignancy) that forms a discrete tumor mass, as opposed to cancers (or malignancies) that diffusely infiltrate a tissue without forming a mass.

In certain embodiments, the cancer is a solid cancer. In some embodiments, the solid cancer is selected in the group consisting of fibrosarcoma, melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma (i.e., hepatocarcinoma), glioblastoma, prostate carcinoma, ovarian cancer and pancreatic carcinoma.

In some embodiments, the age-related disease is selected in the group comprising or consisting of Hutchinson Gilford progeria, Werner syndrome, Alzheimer's disease and cancer.

In some embodiments, the age-related disease is selected in the group comprising or consisting of Hutchinson Gilford progeria, Werner syndrome, Alzheimer's disease and fibrosarcoma.

In some embodiments, the age-related disease is selected in the group comprising or consisting of Hutchinson Gilford progeria, Werner syndrome, Alzheimer's disease and hepatocarcinoma.

In one embodiment, the age-related disease is hepatocarcinoma. In one embodiment, the age-related disease is fibrosarcoma.

In certain embodiments, the progeria is the Hutchinson Gilford progeria.

In one embodiment, the age-related disease is Werner syndrome.

In some embodiments, the nucleic acid molecule, the polypeptide, the vector, the suspension, or the pharmaceutical composition according to the invention is to be administered to an individual in need thereof by any suitable route, i.e., by a dermal administration, by an oral administration, a topical administration or a parenteral administration, e.g., by injection, including a sub-cutaneous administration, a venous administration, an arterial administration, in intra-muscular administration, an intra-ocular administration and an intra-auricular administration.

In certain embodiments, the nucleic acid molecule, the polypeptide, the vector, the suspension, or the pharmaceutical composition according to the invention is to be administered to an individual in need thereof by a dermal administration. In certain embodiments, the nucleic acid molecule, the polypeptide, the vector, the suspension, or the pharmaceutical composition according to the invention is associated with a composition enabling and/or facilitating dermal administration, e.g., by increasing dermal tropism or by increasing dermal barrier penetration. In some embodiments, the dermal administration enables a prolonged liberation of the nucleic acid molecule, the polypeptide, the vector, the suspension, or the pharmaceutical composition according to the invention.

In certain embodiments, the nucleic acid molecule, the polypeptide, the vector, the suspension, or the pharmaceutical composition according to the invention is to be administered to an individual in need thereof by an intravenous administration, in particular by intravenous infusion or intravenous injection.

Within the scope of the instant invention, the therapeutically effective amount of the nucleic acid molecule, the polypeptide, the vector, the suspension, or the pharmaceutical composition according to the invention, to be administered may be determined by a physician or an authorized person skilled in the art and can be suitably adapted within the time course of the treatment.

In certain embodiments, the therapeutically effective amount to be administered may depend upon a variety of parameters, including the material selected for administration, whether the administration is in single or multiple doses, and the individual's parameters including age, physical conditions, size, weight, gender, and the severity of the age-related disease to be treated.

In certain embodiments, a therapeutically effective amount of the isolated polypeptide, or the pharmaceutical composition comprising the isolated polypeptide according to the invention, agent may range from about 0.001 mg to about 3,000 mg, per dosage unit, preferably from about 0.05 mg to about 100 mg, per dosage unit.

Within the scope of the instant invention, the expression “from about 0.001 mg to about 3,000 mg” includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the isolated polypeptide or the pharmaceutical composition comprising the isolated polypeptide according to the invention, may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day. Within the scope of the instant invention, the expression “from about 0.001 mg/kg to about 100 mg/kg” includes about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg and 100 mg/kg.

In some embodiments, the therapeutically efficient amount of the isolated nucleic acid molecule, vector, or pharmaceutical composition according to the invention is ranging from about 10¹ to about 10¹⁵ copies per ml. In practice, a therapeutically efficient amount includes about 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴ and 10¹⁵ copies per ml. In certain embodiments, the therapeutically efficient amount is from about 10¹ to about 10¹⁵ copies per cm³, which includes about 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴ and 10¹⁵ copies per cm³. In some embodiments, the therapeutically efficient amount is from about 10¹ to about 10¹⁵ copies per dose, which includes about 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴ and 10¹⁵ copies per dose.

In certain embodiments, the isolated nucleic acid molecule, isolated polypeptide, vector, suspension, or pharmaceutical composition according to the instant invention is to be co-administered, or sequentially administered, with a drug suitable for preventing and/or treating an age-related disease, in particular a disease selected in the group consisting of progeria, Werner syndrome, neurodegenerative disease, Alzheimer's disease, cancer, cardiovascular disease, obesity, type 2 diabetes, hypercholesterolemia, hypertension, ocular disorders, cataracts, glaucoma, osteoporosis, blood clotting disorders, arthritis, hearing loss and stroke.

As used herein, the term “co-administered” refers to a simultaneous administration of the active principles. As used herein, the term “sequentially administered” refers to an administration of a first active principle before or after the administration of a second active principle.

Another aspect of the invention relates to a kit comprising (i) an isolated nucleic acid, an isolated polypeptide molecule, a vector, or a suspension according to the instant invention, and (ii) means to administer the isolated nucleic acid molecule, the isolated polypeptide, the vector, or the suspension.

In some embodiments, the means to administer the isolated nucleic acid molecule, the isolated polypeptide, the vector, or the suspension include a syringe or a catheter.

Another aspect of the invention relates to a method for increasing adipogenic differentiation in a subject, comprising administering an effective amount of a polypeptide having at least 75%, 80%, 85%, 90%, 95%, 96%, 97%, 98%, 99%, 99.5 or 100% sequence identity with the amino acid sequence selected from the group comprising or consisting of SEQ ID NO: 2, SEQ ID NO: 3 and SEQ ID NO: 4, or a nucleic acid molecule encoding thereof. In some embodiments, the nucleic acid molecule has at least 75%, 80%, 85%, 90%, 95%, 96%, 97%, 98%, 99%, 99.5 or 100% sequence identity with the nucleic acid sequence selected from the group comprising or consisting of SEQ ID NO: 6, SEQ ID NO: 7 and SEQ ID NO: 8.

Another aspect of the invention relates to an in vitro method for increasing adipogenic differentiation of a cell, or population of cells, comprising exposing the cell, or population of cells, to an effective amount of a polypeptide having at least 75%, 80%, 85%, 90%, 95%, 96%, 97%, 98%, 99%, 99.5 or 100% sequence identity with the amino acid sequence selected from the group comprising or consisting of SEQ ID NO: 2, SEQ ID NO: 3 and SEQ ID NO: 4, or a nucleic acid molecule encoding thereof. In some embodiments, the nucleic acid molecule has at least 75%, 80%, 85%, 90%, 95%, 96%, 97%, 98%, 99%, 99.5 or 100% sequence identity with the nucleic acid sequence selected from the group comprising or consisting of SEQ ID NO: 6, SEQ ID NO: 7 and SEQ ID NO: 8. In some embodiments, the cell, or population of cells, is a pre-adipocyte.

Another aspect of the invention relates to a method for decreasing α-SMA expression in a subject, comprising administering an effective amount of a polypeptide having at least 75%, 80%, 85%, 90%, 95%, 96%, 97%, 98%, 99%, 99.5 or 100% sequence identity with the amino acid sequence selected from the group comprising or consisting of SEQ ID NO: 2, SEQ ID NO: 3 and SEQ ID NO: 4, or a nucleic acid molecule encoding thereof. In some embodiments, the nucleic acid molecule has at least 75%, 80%, 85%, 90%, 95%, 96%, 97%, 98%, 99%, 99.5 or 100% sequence identity with the nucleic acid sequence selected from the group comprising or consisting of SEQ ID NO: 6, SEQ ID NO: 7 and SEQ ID NO: 8.

Another aspect of the invention relates to an in vitro method for decreasing α-SMA expression in a cell, or population of cells, comprising exposing the cell, or population of cells, to an effective amount of a polypeptide having at least 75%, 80%, 85%, 90%, 95%, 96%, 97%, 98%, 99%, 99.5 or 100% sequence identity with the amino acid sequence selected from the group comprising or consisting of SEQ ID NO: 2, SEQ ID NO: 3 and SEQ ID NO: 4, or a nucleic acid molecule encoding thereof. In some embodiments, the nucleic acid molecule has at least 75%, 80%, 85%, 90%, 95%, 96%, 97%, 98%, 99%, 99.5 or 100% sequence identity with the nucleic acid sequence selected from the group comprising or consisting of SEQ ID NO: 6, SEQ ID NO: 7 and SEQ ID NO: 8. In some embodiments, the cell, or population of cells, is a pre-adipocyte.

Another aspect of the invention relates to a method for decreasing collagen expression in a subject, comprising administering an effective amount of a polypeptide having at least 75%, 80%, 85%, 90%, 95%, 96%, 97%, 98%, 99%, 99.5 or 100% sequence identity with the amino acid sequence selected from the group comprising or consisting of SEQ ID NO: 2, SEQ ID NO: 3 and SEQ ID NO: 4, or a nucleic acid molecule encoding thereof. In some embodiments, the nucleic acid molecule has at least 75%, 80%, 85%, 90%, 95%, 96%, 97%, 98%, 99%, 99.5 or 100% sequence identity with the nucleic acid sequence selected from the group comprising or consisting of SEQ ID NO: 6, SEQ ID NO: 7 and SEQ ID NO: 8.

In some embodiments, collagen is selected from the group comprising or consisting of type I collagen, type II collagen, type III collagen, type V collagen and type IX collagen. In some embodiments, collagen is type I collagen. In some embodiments, the type I collagen is type I alpha 1 collagen or type I alpha 2 collagen. In some embodiments, the type I collagen is type I alpha 1 collagen, and is encoded by COL1A1 gene.

Another aspect of the invention relates to an in vitro method for decreasing collagen expression in a cell, or population of cells, comprising exposing the cell, or population of cells, to an effective amount of a polypeptide having at least 75%, 80%, 85%, 90%, 95%, 96%, 97%, 98%, 99%, 99.5 or 100% sequence identity with the amino acid sequence selected from the group comprising or consisting of SEQ ID NO: 2, SEQ ID NO: 3 and SEQ ID NO: 4, or a nucleic acid molecule encoding thereof. In some embodiments, the nucleic acid molecule has at least 75%, 80%, 85%, 90%, 95%, 96%, 97%, 98%, 99%, 99.5 or 100% sequence identity with the nucleic acid sequence selected from the group comprising or consisting of SEQ ID NO: 6, SEQ ID NO: 7 and SEQ ID NO: 8. In some embodiments, the cell, or population of cells, is a pre-adipocyte.

In some embodiments, collagen is selected from the group comprising or consisting of type I collagen, type II collagen, type III collagen, type V collagen and type IX collagen. In some embodiments, collagen is type I collagen. In some embodiments, the type I collagen is type I alpha 1 collagen or type I alpha 2 collagen. In some embodiments, the type I collagen is type I alpha 1 collagen, and is encoded by COLIA1 gene.

### EXAMPLES

The present invention and disclosure are further illustrated by the following examples.

### Example 1

### 1. Materials and Methods

### 1.1. Study Subjects and Sample Collection

This study group consisted of 1,068 Ashkenazi Jewish (AJ) samples: 496 AJ centenarians and 572 AJ controls that were previously collected as part of longevity study at the Albert Einstein College of Medicine. A centenarian is defined as a healthy individual living independently at 95 years of age or older and a control is defined as an individual without a family history of unusual longevity; parents of controls survived to the age of 85 years or less. Informed written consent was obtained in accordance with the policy of the Committee on Clinical Investigations of the Albert Einstein College of Medicine. Genomic DNAs were extracted from blood samples and amplified using Illustra GenomiPhi V2 DNA Amplification kits (GE healthcare Life Sciences®).

### 1.2. Two Rounds of Targeted Sequencing

In stage 1, 301 candidate genes involved in genome maintenance were selected and sequenced in 51 centenarians and 51 controls. The complete list of 301 candidate genes is provided in Table 1.

TABLE 1
List of 301 genome maintenance genes sequenced in first round

AATF
AICDA
ALKBH2
ANAPC10
ANAPC11
ANAPC13
ANAPC4
ANAPC5
ANAPC7
APC
APC2
APEX1
APEX2
ATM
ATMIN
ATR
ATRIP
ATRX
BARD1
BLM
BRCA1
BRCA2
BRCC3
BRIP1
BTRC
BUB1
C19orf40
CBL
CDC16
CDC20
CDC23
ERCC8
EXO1
FAM175A
FANCA
FANCB
FANCC
FANCD2
FANCE
FANCF
FANCG
FANCI
FANCL
FANCM
FBXO4
FEN1
GABARAPL2
GADD45A
GADD45B
GTF2H1
GTF2H2
GTF2H3
GTF2H5
H2AFX
HDAC9
HERC1
HERC2
HLTF
HMGB1
HUS1
HUWE1
ITCH
LIG1
LIG3
LIG4
LMNA
MBD4
MDM2
MEN1
MGMT
MGRN1
MID1
MLH1
MLH3
MNAT1
MPG
MRE11A
MSH2
MSH3
MSH4
MSH6
MUTYH
NBN
NEDD4
NEIL1
NEIL2
NEIL3
NHEJ1
NHLRC1
NTHL1
NUDT1
OGG1
PALB2
PAPD7
PARK2
PARP1
PARP2
PCNA
PIAS1
PIAS4
PINX1
PML
PMS1
PMS2
PNKP
POLA1
POLB
POLD1
POLD2
POLE
POLE3
POLE4
POLG
POLH
POLI
POLK
POLL
POLM
POLN
POLQ
POT1
POU1F1
POU2F1
POU2F2
POU2F3
POU3F1
POU3F2
POU3F3
POU3F4
POU4F1
POU4F2
POU4F3
POU5F2
POU6F1
POU6F2
PPIL2
PRDX2
PRKDC
PRPF19
RAD1
RAD17
RAD18
RAD21
RAD23A
RAD23B
RAD50
RAD51
RAD51B
RAD51C
RAD51D
RAD52
RAD9A
RAG1
RBX1
RCHY1
RECQL
RECQL4
RECQL5
REV1
REV3L
RFC1
RFC2
RFC3
RFC4
RFC5
RFWD2
RNF168
RNF7
RNF8
RPA1
RPA2
RPA3
RPA4
SAE1
SCARA3
SENP1
SENP2
SENP3
SENP5
SENP6
SENP7
SENP8
SIAH1
SIRT1
SIRT2
SIRT3
SIRT4
SIRT5
SIRT6
SIRT7
SKP1
SKP2
SLC25A24
SMEK1
SMUG1
STRAP
STUB1
SUMO1
SUMO1P1
SUMO2
SUMO3
SUMO4
SYVN1
TBP
TCEB1
TCEB2
TDG
TDP1
TERF1
TERF2
TERF2IP
TERT
TNKS
TP53
TP53BP1
TPP1
TREX1
TREX2
TRIM32
TRIP12
TXN
UBA1
UBA2
UBA3
UBE2A
UBE2B
UBE2C
UBE2D2
UBE2F
UBE2G1
UBE2G2
UBE2H
UBE2I
UBE2J1
UBE2J2
UBE2K
UBE2L3
UBE2L6
UBE2M
UBE2N
UBE2O
UBE2Q1
UBE2S
UBE2T
UBE2U
UBE2V2
UBE2W
UBE2Z
UBE3A
UBE3B
UBE3C
UBE4A
UBE4B
UBOX5
UBR5
UIMC1
UNG
URM1
WRN
WWP1
WWP2
XAB2
XPA
XPC
XRCC1
XRCC2
XRCC3
XRCC4
XRCC5
XRCC6

SureSelectXT Target enrichment platform (Agilent®) was applied to design and capture genomic regions covering exons, exon-intron junctions, and 2 kb proximal promoter sequences of the candidate genome maintenance genes. Individually indexed libraries were sequenced by the Illumina® HiSeq2000. Sequencing reads were aligned using BWA and variants were called by GATK. All variants in the 102 samples were characterized and functionally annotated using ANNOVAR. To prioritize candidate genes, UMINP (Univariate minP) gene-based association with permutation (n=100) was performed, which considered the effect of at least one significant single variant and identified 122 genes showing a nominal p-value less than 0.05 (Table 2).

TABLE 2
List of 122 genes showing significant association
with longevity in gene-based UMINP analysis

| Gene | p-value |
|---|---|
| AATF | <0.01 |
| AICDA | <0.01 |
| ANAPC11 | <0.01 |
| ANAPC5 | <0.01 |
| APC2 | <0.01 |
| APEX2 | <0.01 |
| ATM | <0.01 |
| ATRX | <0.01 |
| BARD1 | <0.01 |
| BLM | <0.01 |
| BRCA1 | <0.01 |
| BUB1 | <0.01 |
| CDC26 | <0.01 |
| CDC34 | <0.01 |
| CUL7 | <0.01 |
| DCLRE1A | <0.01 |
| DCLRE1B | <0.01 |
| DCLRE1C | <0.01 |
| DDB1 | <0.01 |
| DMC1 | <0.01 |
| E2F1 | <0.01 |
| EXO1 | <0.01 |
| FAM175A | <0.01 |
| FANCB | <0.01 |
| FANCD2 | <0.01 |
| FANCF | <0.01 |
| FANCI | <0.01 |
| FANCM | <0.01 |
| FEN1 | <0.01 |
| GADD45B | <0.01 |
| HERC2 | <0.01 |
| HMGB1 | <0.01 |
| HUS1 | <0.01 |
| HUWE1 | <0.01 |
| ITCH | <0.01 |
| LIG1 | <0.01 |
| MBD4 | <0.01 |
| MDM2 | <0.01 |
| MEN1 | <0.01 |
| MGMT | <0.01 |
| MGRN1 | <0.01 |
| MNAT1 | <0.01 |
| MPG | <0.01 |
| MRE11A | <0.01 |
| MSH3 | <0.01 |
| NEIL3 | <0.01 |
| NTHL1 | <0.01 |
| OGG1 | <0.01 |
| PALB2 | <0.01 |
| PINX1 | <0.01 |
| PMS1 | <0.01 |
| POLA1 | <0.01 |
| POLD1 | <0.01 |
| POLE | <0.01 |
| POLH | <0.01 |
| POLM | <0.01 |
| POLN | <0.01 |
| POT1 | <0.01 |
| POU2F1 | <0.01 |
| POU2F3 | <0.01 |
| POU3F3 | <0.01 |
| POU3F4 | <0.01 |
| POU4F2 | <0.01 |
| POU4F3 | <0.01 |
| POU5F2 | <0.01 |
| PPIL2 | <0.01 |
| PRKDC | <0.01 |
| RAD17 | <0.01 |
| RAD23B | <0.01 |
| RAD50 | <0.01 |
| RAD51 | <0.01 |
| RAD51D | <0.01 |
| RAD52 | <0.01 |
| RAD9A | <0.01 |
| RAG1 | <0.01 |
| RBX1 | <0.01 |
| RECQL4 | <0.01 |
| RFC5 | <0.01 |
| RNF168 | <0.01 |
| RNF8 | <0.01 |
| SAE1 | <0.01 |
| SENP1 | <0.01 |
| SENP8 | <0.01 |
| SIRT1 | <0.01 |
| SIRT2 | <0.01 |
| SIRT4 | <0.01 |
| SIRT7 | <0.01 |
| SKP2 | <0.01 |
| STRAP | <0.01 |
| SUMO1 | <0.01 |
| SUMO2 | <0.01 |
| SYVN1 | <0.01 |
| TERF2 | <0.01 |
| TNKS | <0.01 |
| TP53BP1 | <0.01 |
| TREX1 | <0.01 |
| UBA3 | <0.01 |
| UBE2B | <0.01 |
| UBE2C | <0.01 |
| UBE2J2 | <0.01 |
| UBE2O | <0.01 |
| UBE2Q1 | <0.01 |
| UBE2U | <0.01 |
| UBE2V2 | <0.01 |
| UBE2W | <0.01 |
| UBOX5 | <0.01 |
| UIMC1 | <0.01 |
| UNG | <0.01 |
| URM1 | <0.01 |
| XPA | <0.01 |
| XPC | <0.01 |
| XRCC1 | <0.01 |
| CDH1 | 0.01 |
| CREBBP | 0.01 |
| PIAS4 | 0.01 |
| SIRT5 | 0.01 |
| ATR | 0.03 |
| SENP3 | 0.03 |
| SIRT6 | 0.03 |
| NBN | 0.04 |
| POU3F2 | 0.04 |
| RFC4 | 0.04 |

Gene ontology (GO) enrichment analysis was implemented using the clusterProfiler package and identified the enrichment of 122 UMINP-significant genes in DNA double-strand breaks repair functions.

In stage 2, 106 candidate genes involved in DNA double-strand breaks repair were selected and sequenced in 496 centenarians and 572 controls. The list of 106 DSB repair genes is provided in Table 3.

TABLE 3
List of 106 DSB repair genes sequenced in second round

APLF
APTX
ATM
BABAM1
BARD1
BLM
BRCA1
BRCA2
CDC7
CHEK1
CHEK2
DCLRE1A
DCLRE1B
DCLRE1C
DDX11
DNA2
EME1
ERCC1
ERCC4
ERCC6
ERCC8
FAN1
FANCB
FANCD2
FANCM
GEN1
GINS2
GINS4
HELB
HELQ
HUS1
INO80
KAT5
LIG3
LIG4
MEIOB
MGMT
MLH1
MMS19
MMS22L
MSH2
MUS81
NHEJ1
NIPBL
NSMCE1
NUCKS1
OGG1
OTUB1
PALB2
PARP1
PARP2
PARP3
PAXIP1
PNKP
POLA1
POLB
POLM
POLQ
POT1
PRDM9
PRKDC
RAD50
RAD51
RAD51AP1
RAD51AP2
RAD51B
RAD51C
RAD51D
RAD52
RAD54B
RAD54L
RBBP8
REC8
RECQL
RECQL5
REV3L
RMI1
RMI2
RNF168
RNF8
RPA3
RTEL1
SETD2
SETMAR
SETX
SFR1
SIRT6
SLX4
SMARCAL1
SMC5
SPIDR
SPO11
SWI5
TDP1
TDP2
TERF2IP
TP53
TP53BP1
UBE2N
WRAP53
WRN
XRCC1
XRCC2
XRCC4
XRCC5
XRCC6

A 25-sample pooling target capture sequencing was performed. Briefly, 25 samples from the same group were pooled together for equimolar concentration and totally 20 pools of centenarians and 23 pools of controls were used to generate indexed libraries according to the Illumina® TruSeq DNA sample preparation low-throughput (LT) protocol. Captured libraries were further generated using SeqCap EZ Choice Enrichment Kit (Roche®) which enriched genomic regions covering exons, exon-intron junctions, and 2 kb proximal promoter sequences of the candidate DSB genes and sequenced by Illumina® HiSeq2000. Sequencing reads were aligned using BWA and variants were called using the software CRISP (Comprehensive Read analysis for Identification of Single Nucleotide Polymorphisms), which was specifically developed to call variants in pooled DNA sequence data. All variants in the 1,068 samples were characterized and functionally annotated using ANNOVAR. To test for longevity associations, we performed different subtypes of SKAT (Sequencing Kernel Association Test) analyses, such as SKAT for rare variants, SKAT-C for all variants (common and rare), and SKAT-O for variants with directionality (enrichment only in centenarians or in controls) (Table 4).

TABLE 4
List of 10 longevity-associated DSB repair genes with
their centenarian-enriched missense variants (part 1)

| Gene | SKAT-C | SKAT-O | SKAT |
|---|---|---|---|
| PARP2 | 0.00142 | 0.01563 | 0.01362 |
| RAD51B | 0.0041 | 0.04227 | 0.025 |
| SIRT6 | 0.03423 | 0.33932 | 0.20718 |
| TERF2IP | 0.042 | 0.00703 | 0.02925 |
| ATM | 0.04499 | 0.05524 | 0.03293 |
| MLH1 | 0.06486 | 0.07838 | 0.03947 |
| BRCA2 | 0.07093 | 0.0099 | 0.03295 |
| ERCC6 | 0.28236 | 0.01455 | 0.01379 |
| MMS22L | 0.39608 | 0.04202 | 0.36627 |
| TP53 | 0.47502 | 0.00147 | 0.00488 |

### 1.3. Cell Lines

HEK293 SIRT6 overexpression lines were generated by transfecting HEK293 cells with linearized CMV-SIRT6 plasmids via jetPRIME transfection reagent and selecting stably integrated clones. To generate normal human fibroblasts expressing WT and centenarian alleles of SIRT6 under control of cumate-inducible promoter (cumate SIRT6 fibroblasts), constructs containing SIRT6 alleles under control of cumate promoter were integrated in the genome of telomerase immortalized SIRT6-KO human HCA2 foreskin fibroblasts via the PiggyBac Transposon Vector System. Endogenous SIRT6 was knocked out in these cells using CRISPR/Cas-9. NHEJ and HR reporter assays were conducted using telomerase-immortalized HCA2 human fibroblast cell lines containing integrated reporter constructs (19A and H15C). HT1080 and HeLa cell lines were used to assess anti-tumor activity.

### 1.4. Cell Culture

All cell lines were maintained in humidified incubators at 5% CO₂, 5% O₂, at 37° C. Cells were grown in Eagle's minimum essential medium with 15% fetal bovine serum and 1× penicillin/streptomycin, with the exception of the HEK293, HT1080 and HeLa cells, which were culture in DMEM with D-Glucose and L-Glutamine. The cell lines are routinely tested for mycoplasma contamination.

### 1.5. Western Blotting

Cells and reactions were collected using a 2× Laemmli solution and incubated on ice for 15 min, during which time the samples were passed through a large gauge needle several times and vortexed every 5 min. Samples were then spun at 14,000 RPM to remove debris and the supernatant transferred to a new tube. Samples were heated in boiling water for 20 min before being centrifuged at 14,000 RPM for 1 min and loaded into a BioRad® Criterion 4-20% gel. After transfer to PDVF membrane and blocked (5% dehydrated milk) for 2 h at RT (room temperature), membranes were incubated overnight antibodies in 2.5% blocking buffer at 4° C. Membranes were washed 3× with TBST for 10 min each before secondary antibody in 1×TBST was added for 2 h incubation at RT. Membranes were washed 3× with TBST for 10 min and then imaged.

The following antibodies were used: H3 (Abcam® ab500)-1:5,000, H3K9ac (Abcam® ab4441)-1:1,000, H3K18ac (Abcam® ab1191)-1:1,000, β-tubulin (Abcam® ab6046)-1:10,000, SIRT6 (Cell Signaling® #12486)-1:1,000, AbD33204 (Rabbit, REF)-1:500, Lamin A/C-1:1,000 (Abcam® ab108595, Millipore® 05-714), γH2AX (Millipore® 05-636), PARP1(Abcam® ab227244)-1:1,000, mADPr 1:500 (AbD33204 and AbD33205).

### 1.6. Immunoprecipitation (IP)

Cells were plated and grown with equivalent dosages of cumate for 48 h prior to IP. In brief, cells were collected via trypsin and centrifugation and then lysed on ice using IP Buffer (20 mM HEPES pH=8, 0.2 mM EDTA, 5% glycerol, 150 mM NaCl, 1% NP40)+COMplete® protease inhibitor for 10 min. Lysate was sonicated at 25% output for 10 pulses and then debris pelleted by centrifugation at 4° C. at 13,000 RPM for 10 min. Supernatant was transferred to a new tube and precleared with A/G Sepharose beads at 4° C. for 1 h on a rotator. Beads were removed by centrifugation and transfer of cleared sample to new tubes. 50 μl of sample was reserved as input control. Samples were incubated overnight at 4° C. with 3 μg of antibody (SIRT6; Cell Signaling®) #12486; PARP1-Abcam® ab227244; Lamin A/C-Millipore® 05-714; AbD33204, AbD33205, then with 30 μl of 25% Sepharose for 2 h at 4° C. Samples were spun down and beads washed 5× with IP buffer. Final resuspension with 100 μl IP buffer.

### 1.7. DNA Repair Assays

Both NHEJ and HR efficiency were assessed as previously described in Seluanov et al. (J Vis Exp, 2010; doi: 10.3791/2002). In brief, I-Scel, SIRT6 or HPRT control, and dsRED plasmid were transfected into telomerase-immortalized human foreskin fibroblasts containing chromosomally integrated NHEJ or HR reporter cassettes (19A or H15C) cells. Cells were allowed 3 days to recover prior to analysis by flow cytometry. Efficiency was calculated as the ratio of GFP events over dsRED events.

### 1.8. Transfections

Transfections were carried out by plating cells at a density of 500,000 cells/10 cm plate two days prior to transfection. Transfections were carried out using the Amaxa® Nucleofector with Normal Human Dermal Fibroblast transfection solution following manufacture's protocol. For cancer cell transfections, jetPRIME transfection reagent was used to deliver plasmids into cells.

### 1.9. Quantitative RT-PCR

Total RNA was isolated from cells at 80% confluence using Trizol Reagent and then treated with DNase. cDNA was synthesized using Superscript III (Life Technologies®) cDNA kit with the OligodT primer. qRT-PCR was performed on the BioRad® CFX Connect Real Time machine with SYBR™ Green Master Mix (BioRad®) using 30 ng of cDNA per reaction with 3× reactions/sample. All primer sets (see Table 5 below) were tested for specificity and efficiency. Actin was assayed using Quantum mRNA Actin Universal primers (Thermo Fisher Scientific® AM1720).

TABLE 5
Primers used for the qRT-PCR

| Primer name | Sequence | SEQ ID NO: |
|---|---|---|
| LINE1 ORF1 Fwd | ATGGCGAAAGGCAAACGTAAG | SEQ ID NO: 9 |
| LINE1 ORF1 Rev | ATTTTCGGTTGTGTTGGGGTG | SEQ ID NO: 10 |
| LINE1 ORF2 Fwd | GCAGGGGTTGCAATCCTAGTC | SEQ ID NO: 11 |
| LINE1 ORF2 Rev | CTGGGTGCTCCTGTATTGGGT | SEQ ID NO: 12 |

### 1.10. Immunofluorescence and Apoptosis

γH2AX immunostaining was carried out as previously described in Mao et al. (Science, 2011; Vol. 332:1443-1446). Anti-γH2AX antibody was purchased from Millipore® (05-636). Apoptosis in fibroblasts was measured using the Annexin V Staining Kit (Roche®).

### 1.11. Gamma Irradiation

Cells were grown to 75% confluency prior to treatment on coated slides. A Cs-137 irradiator was used to deliver 2 Gy of radiation to cells. Cells were transported in a 37° C. container and media were replaced post exposure.

### 1.12. Paraquat Treatment

Cells were maintained to 75% confluency, at which point fresh media lacking sodium pyruvate and containing paraquat was added. Cells were maintained for 24 h in treated media, followed by replacement with fresh media lacking sodium pyruvate. 48 h post treatment, the cells stained and evaluated for apoptosis.

### 1.13. SIRT6 Protein Purification

His-tagged SIRT6 cDNA was cloned into pET11a vectors and transformed into Rosetta-Gami E. coli cells. Cells were grown in presence of antibiotics and then protein production was induced with 0.5 mM IPTG 2 h before harvest. Cells were spun down and lysed on ice for 1 h using a in a solution of 50 mM Tris-HCl (pH=7.5), 300 mM NaCl, 10% glycerol, and 10 mM imidazole with EDTA-free protease inhibitor (Sigma Aldrich®, #P8849) and 1 mg/ml egg white lysozyme followed by sonication with a Branson instrument. After removal of cell debris by centrifugation, lysate was incubated with Ni2+-NTA agarose overnight. Solution with beads was placed in gravity column and washed with lysis solution, followed by 2 volumes of wash buffer (lysis buffer+30 mM imidazole). Finally, protein was eluted with elution buffer (lysis buffer+500 mM imidazole) and fractions were collected. Protein concentration was assessed by bicinchoninic acid (BCA) assay and run on an SDS-page gel. The 3-4 highest concentration fractions were pooled and dialyzed against storage buffer (50 mM Tris-HCl pH=7.5, 150 mM NaCl, 1 mM DTT, 5% glycerol).

### 1.14. Stable Isotope Labeling of Amino Acids in Cell Culture (SILAC)

Turnover experiments were performed using HEK293 cell lines stably expressing SIRT6 WT or centSIRT6. Cells were cultured for one week until confluent in MEM for SILAC (Thermo Fisher Scientific®) supplemented with 10% dialyzed FBS (Gibco®), L-glutamine, L-arginine ¹³C₆ ¹⁵N₄ (Cambridge Isotopes®), and L-lysine ¹³C₆ ¹⁵ N₂ (Cambridge Isotopes®). The media were changed to normal culture medium and cell pellets were harvested at 0, 2, 4, 6, 8, 12, and 24 h post-media change. Cell pellets were lysed in buffer containing 8 M urea, 75 mM NaCl, 50 mM Tris, pH 8.5, with protease inhibitor cocktail (Roche®). Pellets were vortexed for 30 s and sonicated 5×10 s with 1 min rest on ice in between sonication steps. Lysate was centrifuged at 15,000×g for 10 min and then supernatant was collected.

### 1.15. SIRT6 Deacylation Activity

The substrate for this reaction is a synthetic TNF-derived peptide first developed by Schuster et al. (Sci Rep, 2016; Vol. 6:22643) was synthesized by Genscript®. Reactions were performed with 150 mM NaCl, 20 mM Tris, 5% glycerol, 1 mM β-mercaptoethanol, 2 μM SIRT6, and substrate concentration ranges of 7-1000 μM NAD+ and 2-88 UM peptide. Reactions were performed at 37° C. and fluorescence was measured using 310/405 nm excitation/emission spectra on a Tecan Spark 20M plate reader. Readings were taken every 30 s and initial rates were calculated from the relative fluorescence increase over a minimum of 6 min.

### 1.16. Histone Analysis

Histones were purified from cumate SIRT6 fibroblasts (SIRT6 KO primary human foreskin fibroblasts constitutively expressing the catalytic subunit of telomerase as well as various alleles of SIRT6 via a cumate-inducible promoter) using an established acid extraction method followed by propionylation of lysines prior to trypsin digestion to enhance coverage. A data independent analysis (DIA) mass spectrometry (MS) method was employed for quantitation of modified peptides after samples were separated by nano-LC using EpiProfile® or, alternatively, after direct infusion followed by a one-minute acquisition and analysis with EpiProfileLite®.

### 1.17. Analysis of SIRT6 and LMNA Protein-Protein Interactions by Mass Spec

Cumate SIRT6 fibroblasts were used to compare interactomes for centSIRT6 and the wild type SIRT6. Nuclear extracts were prepared using hypotonic lysis buffer. Nuclei isolated from approximately 2.5×10⁷ cells were resuspended in 1 ml of extraction buffer (50 mM TrisHCl pH 7.5, 150 mM NaCl, 10% glycerol, 5 mM DTT, 0.25% NP-40, 1× Roche® Complete protease inhibitors, as well as kinase/phosphatase inhibitors-1 mM NaVO₄, 10 mM beta-glycerophosphate, 1 mM sodium pyrophosphate, 1 mM NaF) and nuclei were disrupted by passage through a 27-gauge needle followed by sonication (3 pulses at constant 20% power) with a Branson® Sonifier on ice. Next, samples were filtered through 0.2 μm MWCO filter to remove any insoluble material (and reduce non-specific binding). Proteins were quantified using the BCA assay and equal amount of wild type SIRT6 and centSIRT6 derived extract were divided into 5 replicates each. Three replicates of extract received 4 μg of anti-SIRT6 antibody (Cell Signaling® #1248) or anti-LaminA (Lamin A/C-Millipore® 05-714) and 75 μl of Miltenyi® protein G uMACS magnetic particles. Two of the replicates were mixed with normal rabbit IgG (Cell Signaling® #2729) as controls for non-specific binding to the particles. Samples were rotated at 4° C. for 6 h followed by separation by the magnet. Samples were washed with 3 additional ml of extraction buffer and eluted with 100 μl of boiling elution buffer (5% SDS with 50 mM Tris-HCl pH 7.5). After removal of SDS with S-columns (Protifi®; Huntington, NY) and trypsin digestion, peptides were resuspended in MS-grade water and labeled with tandem mass tags (TMT 10-plex; Thermo Fisher Scientific®). Samples were resolved by nano-electrospray ionization on an Orbitrap Fusion Lumos MS instrument (Thermo Fisher Scientific®) in positive ion mode using a 30 cm home-made column packed with 1.8 μm C-18 beads to resolve the peptides. Solvent A was 0.1% formic acid and solvent B was 100% acetonitrile (CAN) with 0.1% formic acid. The length of the run was 2 h with a 90 min gradient. CID (35% collision energy) was used for MS2 fragmentation. HCD (60% collision energy) was used for MS3 detection of TMT groups. Other details of the run parameters may be found in the embedded run report of the RAW data file uploaded to the ProteomeXchange database. Peptide assignments were made using Proteome Discoverer and Sequest and MS3 ions were used for quantitation. False discovery rates (FDR) were estimated using a Decoy Database Search with Target FDR (strict) set to 0.01 and Target FDR (relaxed) set to 0.05. Validation was based on q-value. In the consensus step, ions with a co-isolation threshold above 30% were excluded. Normalization between replicates was achieved using the total protein approach where peptide counts for a single protein were divided by the sum of all proteins in the lane. Proteins appearing in specific antibodies (either anti-SIRT6 or anti-Lamin A) compared to normal IgG serum with Student t-test p<0.05) were considered as interactions. Similarly, proteins showing higher levels in centSIRT6 versus wild type SIRT6 were based upon p<0.05.

### 1.18. SIRT6-NAD+ Binding by Tryptophan Fluorescence

1 μM of SIRT6 protein in 50 μl of buffer (50 mM Tris-HCl pH=7.5 and 150 mM NaCl) was combined with a range of NAD+(0-2 mM). Samples were then placed in a 384 Corning® Flat Black plate and analyzed in a Tecan Spark® 20M plate reader. Fluorescence was measured across 300-400 nm range in 2 nm steps using a 280 nm excitation. Background quenching by NAD+ of SIRT6 denatured by 7 M urea was subtracted from the spectra as described previously in Pan et al. (J Phys Chem B, 2011; Vol. 115:3245-3253).

### 1.19. Deacetylase Assay

3 μg of SIRT6 protein was combined with 0.5 μg histones, 1 or 5 mM NAD+, 30 mM Tris-HCl pH=8, 4 mM MgCl₂, 1 mM DTT and ddH₂O up to 50 μl. All reagents were prepared on ice and moved to 37° C. for the duration of the incubation. Reaction was quenched with direct application of 1 volume of 2× Laemmli buffer with BME. Samples were boiled for 15 min before being run in Western analysis using anti-H3K9ac and anti-H3K18ac antibodies.

### 1.20. PARP1 Ribosylase Assay

5 μg of SIRT6 protein was combined with 100 fm PARP1, 20 mM Tris-HCl pH=8, 10 μM ZnCl₂, 10 μM MgCl₂, 10% glycerol, 300 μM NAD+, 1 mM DTT, 0.1 μg/ml salmon sperm DNA and ddH₂O up to 50 μl. All reagents were prepared on ice and moved to 30° C. for 30 min. Reaction was quenched with direct application of 1 volume of 2× Laemmli buffer with BME. Samples were boiled for 15 min before being run in Western analysis using anti-PADPR antibody.

### 1.21. Self-Ribosylation Assay

Three μg of SIRT6 was combined with 50 mM Tris-HCl pH=7.5, 1 mM DTT, 10 μM ZnCl₂, 150 mM NaCl, 25 μM NAD+, 1 μl [32P]-NAD+(800 Ci/mmol, 5m Ci/ml; Perkin Elmer® BLU023x250UC) and ddH₂O up to 20 μl. All reagents were combined on ice in a master mix except for SIRT6. Master mix was aliquoted into reaction tubes and SIRT6 was added and incubated at 37° C. for 3 h. Reaction were quenched with 1 volume of 2× Laemmli buffer with BME. Samples were boiled and then run on SDS-PAGE gel and then transferred to PDPF membrane. Self-ribosylation was assayed using phosphoimager.

### 1.22. Thermostability

4 μM SIRT6 protein was combined with 1× SYPRO™ Orange dye in storage buffer (50 mM Tris-HCl pH=7.5, 150 mM NaCl, 1 mM DTT, 5% glycerol) to 50 μl. Samples were placed in qRT-PCR plate and run on a BioRad® CFX Connect Real Time machine using a Melt Curve protocol (30° C.-75° C., 0.5° C. steps in 10 s intervals).

### 1.23. FRET

Mutagenesis to generate the centenarian mutant (N308K/A313S) was performed using NEB Q5 site-directed mutagenesis kit, following protocols for primer design.

HEK293-6E cells were transfected using 293fectin® reagent according to the manufacturer's instructions (Thermo Fisher Scientific®). The SIRT6 biosensors were expressed using mammalian expression vector pTT5 (National Research Council, Canada). After two days in culture, approximately 30 million were produced, generating transiently transfected cell lines expressing the biosensor at high levels. The cell line was maintained using F17 medium (Sigma Aldrich®)+(200 nM/mL) GlutaMAX®.

On each day of FRET assays, approximately 30 million cells from each transfection were harvested, washed three times in PBS with no Mg or Ca (Thermo Fischer Scientific®; Waltham, MA) by centrifugation at 300×g, filtered using 70-μm cell strainers (Corning®; Corning, NY), and diluted to 1-2 million cells/mL using a Countess™ Automated cell counter (Invitrogen®; Carlsbad, CA). On each day of experiments, cell viability was assessed using the trypan blue assay. After resuspension and dilution in PBS, the cells were continuously and gently stirred with a magnetic bar at room temperature to keep the cells in suspension and prevent clumping.

An in-depth description of the fluorescence instrumentation has been published previously in Schaaf et al. (SLAS Discov, 2017; Vol. 22:262-273). For FLT measurements, the observed fluorescence waveform was convolved with the instrument response function to determine F(t) (Equation 1), the average energy transfer efficiency E was calculated from the average FLTs of donor ID and donor-acceptor, IDA, using Equation 2, and the donor-acceptor distance R was calculated using Equation 3.

$$F ( t ) = {\sum }_{i = 1}^{2} {A}_{i} exp ( \frac{- t}{{τ}_{i}} ) \qquad ( Equation 1 )$$

$$F R E T E = 1 - \frac{{τ}_{D A}}{{τ}_{D}} \qquad ( Equation 2 )$$

$$R = {{R}_{0} [ ( 1 / E ) - 1 ]}^{1 / 6} \qquad ( Equation 3 )$$

### 1.24. Quantification and Statistical Analysis

Unless otherwise noted, the Student's t-test was used to determine statistical significance between groups. All tests were two-tailed and p-values were considered significant below a 0.05 threshold. Cell culture experiments utilized at least two separately derived cell lines for each genotype and were performed in triplicate unless noted otherwise. In vitro assays were done in triplicate using two or more separate protein isolation preps.

### Results

While there is evidence that genome maintenance (GM) is a major longevity assurance system, with genetic defects in GM genes leading to reduced life span and premature ageing in humans and mice and a correlation of more efficient DNA repair with longer maximum lifespan across animal species, the role of GM in human extreme longevity has never been systematically studied.

To investigate whether GM plays a role in human extreme longevity, a panel of 301 genome maintenance genes (Table 1) in pathways including nucleotide excision repair (NER), base excision repair (BER), mismatch repair (MMR), homologous recombination (HR), non-homologous end-joining (NHEJ), telomere maintenance, and genes involved in the regulation of these pathways, was assembled.

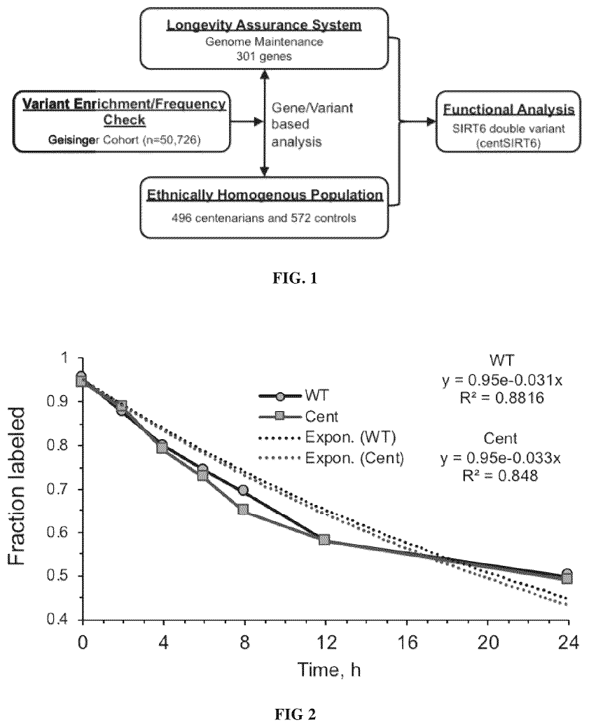

Two rounds of targeted sequencing were then performed to identify rare variants in these genes in DNA from peripheral blood of Ashkenazi Jewish (AJ) centenarians and AJ individuals between 60 and 70, unrelated to the probands and without centenarian relatives (FIG. 1). In the first round, 51 AJ centenarians and 51 AJ controls were sequenced and identified 122 genes that showed association with longevity (Table 2). Gene ontology analysis showed that these genes were enriched for DNA double strand breaks (DSB) repair functions. DSB repair genes were next sequenced in a larger population of 496 AJ centenarians and 572 controls (Table 4).

SIRT6, a gene functionally related not only to DSB repair but also to longevity in model organisms was especially interesting. SIRT6 knockout mice show premature ageing and genomic instability whereas mice overexpressing SIRT6 display an extended lifespan (see Mostoslavsky et al.; Cell, 2006; Vol. 124:315-329). Higher SIRT6 activity is associated with more efficient DSB repair and longer maximum lifespan across mammalian species. At the molecular level, SIRT6 is involved in DNA repair, telomere maintenance, silencing of the repetitive elements including LINE1 retrotransposons, regulation of glucose homeostasis, inflammation and pluripotency, and tumor suppression.

The SIRT6 allele found enriched in centenarians contained two missense mutations within the highly flexible C-terminus, converting a polar asparagine 308 to a charged lysine (N308K) and a hydrophobic alanine 313 to a polar serine (A313S). Genotyping in centenarian carriers confirmed that these two SIRT6 variants occurred as linked double variants, which we named centSIRT6. This allele was very rare (minor allele frequency of 0.001%) in the whole exome sequence data of 50,726 adult participants of predominantly European ancestry in the Geisinger Health System. It was next tested whether centenarian SIRT6 allele alters the biological activities of SIRT6 protein.

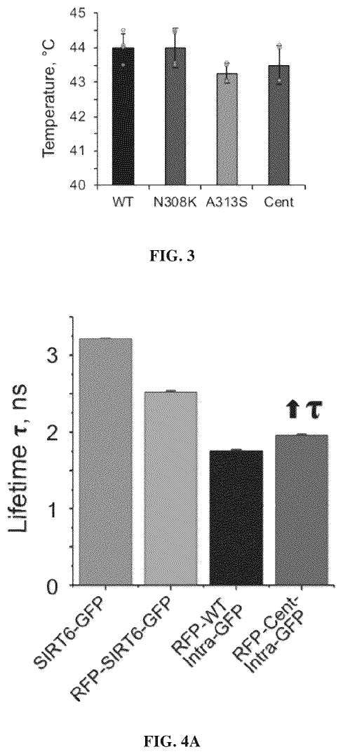

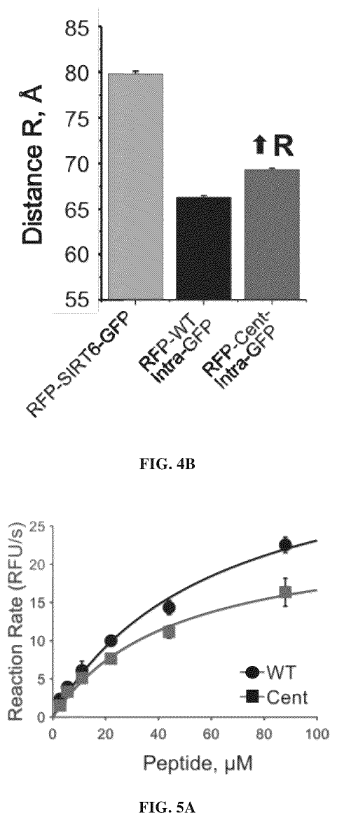

SILAC analysis of HEK293 cells expressing different SIRT6 alleles showed no difference in the protein turnover rates between the WT and centSIRT6 (FIG. 2). To assess the biochemical properties of the different SIRT6 alleles, recombinant versions of each allele were purified. Consistent with similar half-lives of wild type and centSIRT6 in vivo, it was observed no notable differences in the thermal stability of the SIRT6 alleles in vitro (FIG. 3), suggesting that centSIRT6 does not grossly alter the folding or the ambient stability. As centSIRT6 mutations change the polarity and charge of the residues, SIRT6 FRET biosensors fused to the middle and the C-terminus of SIRT6 were used to assess for more subtle changes to protein structure that centSIRT6 mutations might cause (FIG. 4A-4B). The centSIRT6 decreased FRET by ΔE=6.4±0.1% relative to the wild type SIRT6, corresponding to an increase in the donor-acceptor distance R by 3.0+0.4 Å. This increase indicates centSIRT6 mutations shift the SIRT6 structure toward a more open conformation.

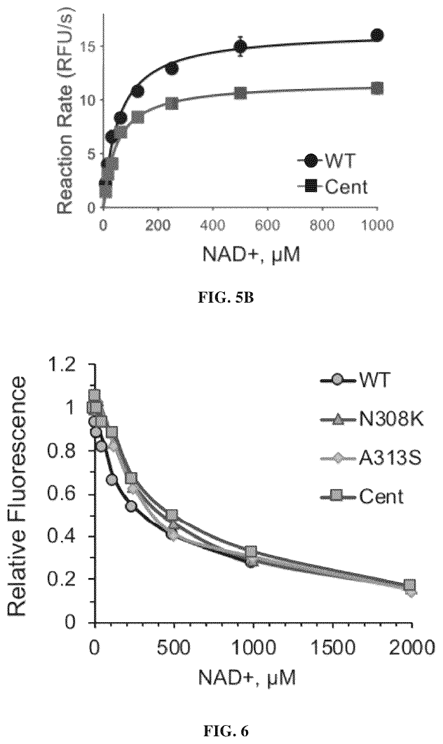

SIRT6 deacylase activity was tested on a myristolated peptide to determine Km for NAD+ and peptide for wild type and centSIRT6. While the centenarian allele displayed a slight reduction in activity (Vmax) compared to the wild type (p=0.02) (FIG. 5A-5B), Km was not significantly different between wild type and centSIRT6 for both substrates (Table 6).

TABLE 6
Kinetic parameters Km and Vmax

| | Km_{NAD} (μM) | Km_{PEP} (μM) | Vmax (RFU/S | Vmax/Km_{PEP} |
|---|---|---|---|---|
| WT | 55.5 ± 5.4 | 61.1 ± 12.4 | 37.2 ± 4.1 | 0.61 |
| Cent | 48.4 ± 3.7 | 46.0 ± 11.3 | 24.3 ± 2.9 | 0.53 |

Quenching of SIRT6 intrinsic Trp fluorescence by NAD+ binding also showed that centSIRT6 has similar affinity for NAD+ to the wild type allele (FIG. 6). Overall, these data indicate that centSIRT6 confers a slight reduction in deacylase catalytic efficiency (as defined by Vmax/Km) (Table 6).

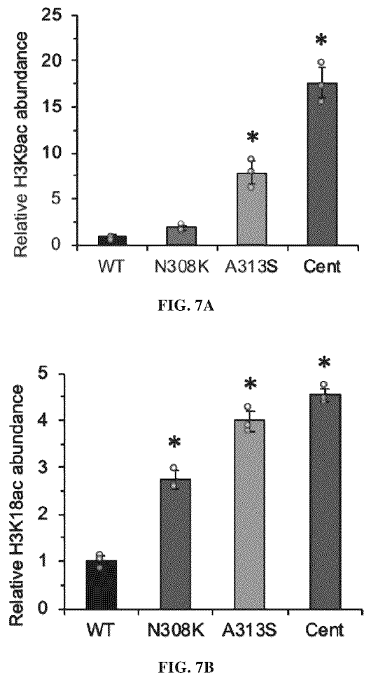

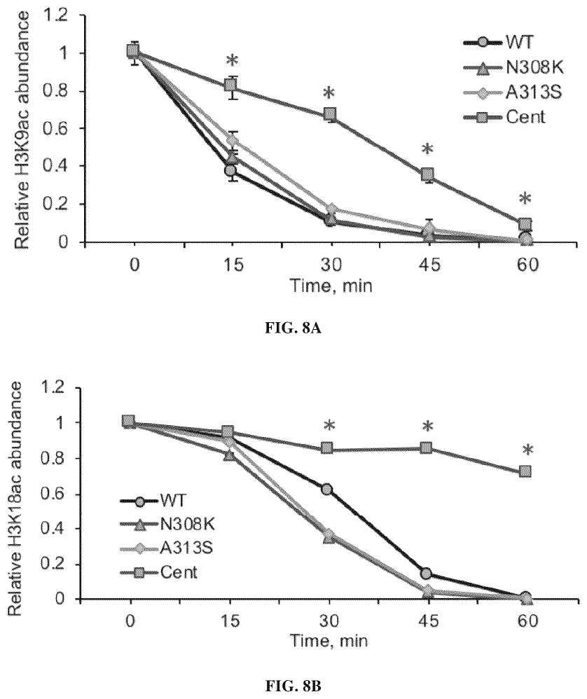

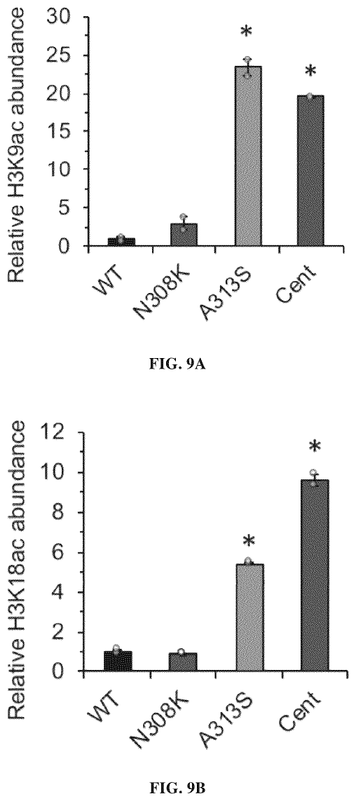

The centSIRT6 displayed significantly lower deacetylase activity on recombinant nucleosomes with saturated H3K9ac and H3K18ac modifications (FIG. 7A-7B). Additionally, centSIRT6 allele displayed significantly slower histone deacetylase kinetics (FIG. 8A-8B). Similar to the synthetic histones, the centSIRT6 allele deacetylated both H3K9ac and H3K18ac residues on nucleosomes purified from Hela cells, at a significantly reduced rate compared to the wild type SIRT6 (FIG. 9A-9B). Taken together, the combined data from deacylase and histone deacetylase experiments indicate that the centSIRT6 variant has reduced deacylase/deacetylase activity.

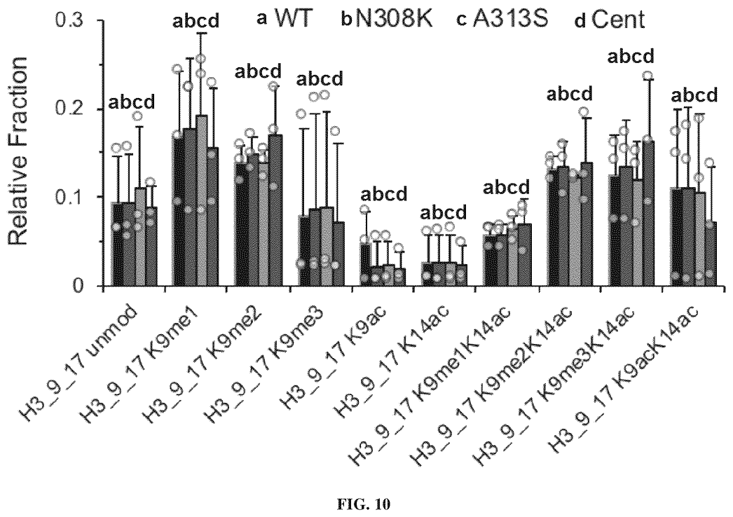

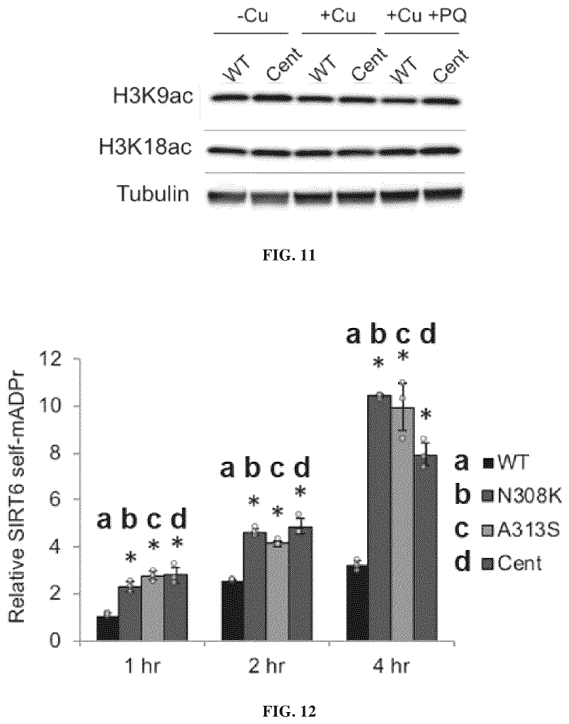

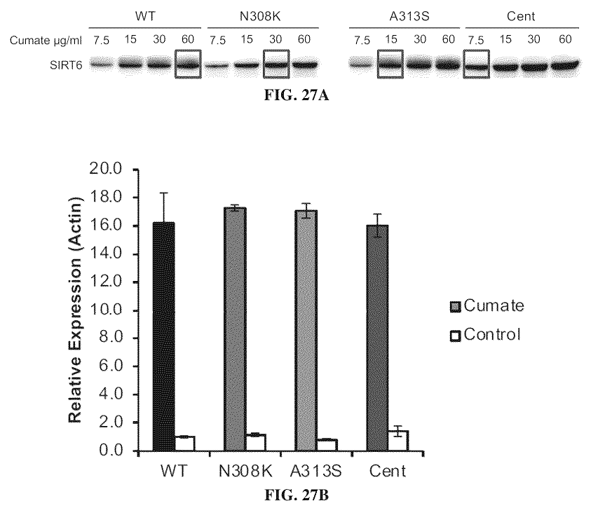

To test the effect of the centSIRT6 allele on histone deacetylation activity in vivo, telomerase-immortalized human HCA2 fibroblast cell lines were generated in a SIRT6 KO background containing a cumate switch promoter driving expression of SIRT6 WT, N308K, A313S, and the centSIRT6 alleles (cumate SIRT6 fibroblasts). Each cell line was induced with cumate to drive equal levels of SIRT6 expression (FIGS. 27A-27B). Histone post-translational modifications from purified histones were quantified from these cells by mass spectrometry using peptide standards as described previously (Sidoli et al. Journal of visualized experiments: JoVE, 2016; doi: 10.3791/54112). Cells expressing the centSIRT6, N308K, and A313S SIRT6 alleles showed similar global histone post translational modification (PTM) levels at all sites known to be SIRT6 substrates including H3K9ac, H3K18ac, and H3K56ac (FIG. 10). Similarly, global levels of H3K9ac and H3K56ac measured by Western blot did not show significant changes, even when challenged with paraquat-induced oxidative damage (FIG. 11). These results suggest that reduced deacylation activity of centSIRT6 is compensated in vivo by additional protein factors or PTMs that help maintain SIRT6 deacetylation activity at close to the wild type levels.

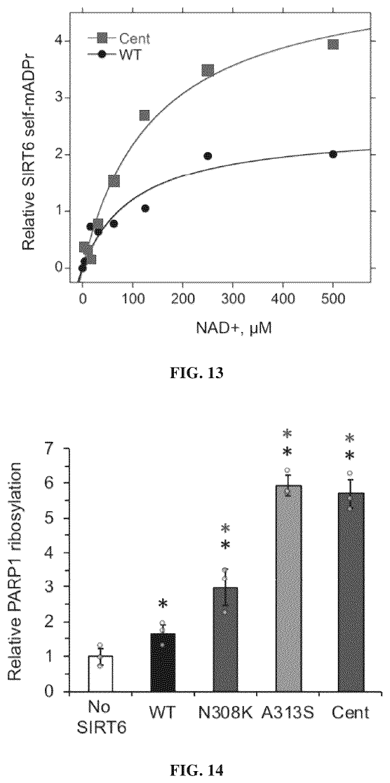

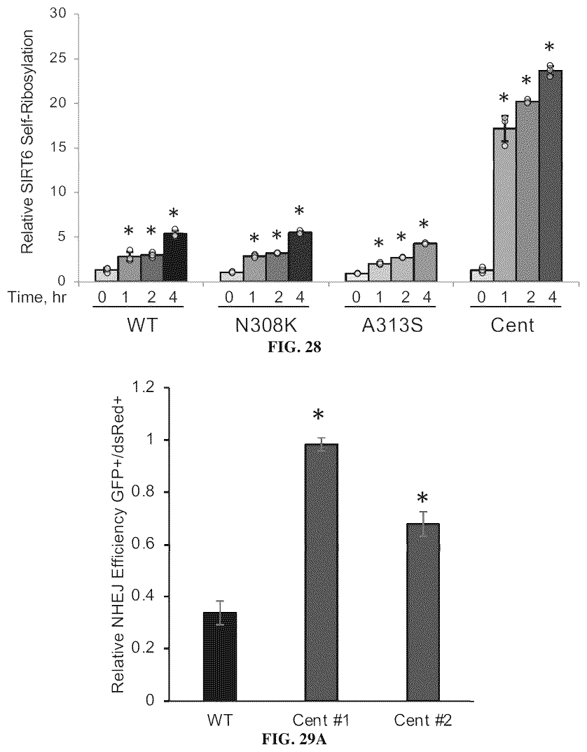

In addition to its deacetylase activity, SIRT6 has mono-ADP ribosylation (mADPr) activity, able to mono-ADP ribosylate itself and other proteins. mADPr activity is critical to SIRT6 role in DNA damage response and LINE1 repression. To assess the mADPr activity, two known SIRT6 substrates were used: PARP1 and SIRT6, itself. The centSIRT6 demonstrated a significantly higher auto-ribosylation rate with radio labeled NAD+ than the wild type SIRT6 (FIG. 12). The single SNP alleles and the combined centSIRT6 displayed a similar increase in self-mADPr compared to the wild type, suggesting a general increase that is not cumulative between the two SNPs (FIGS. 12 and 28). Following this, an antibody with specificity to mADPr residues was utilized to examine the self-ribosylation efficiency using a titration of NAD+(Bonfiglio et al.; Cell, 2020; Vol. 183:1086-1102). As with the radiolabeled NAD+, centSIRT6 displayed almost 2-fold higher maximal mADPr activity and displayed a higher Km NAD of 142+29, compared to 106+45 for wild type SIRT6 (FIG. 13). Thus, the overall efficiency as defined by mADPrmax/Kapp NAD was 0.037 for centSIRT6 compared to 0.023 for wild type SIRT6, which also equates to a nearly two-fold difference. In trans, mADPr activity was assessed by incubation with human PARP1, which was previously shown to be ribosylated by SIRT6. Similar to self-ribosylation, the centSIRT6 displayed higher PARP1 ribosylation activity (FIG. 14). Curiously, while both single mutants had elevated activity, the A313S allele was more similar to the centSIRT6 than N308K (FIG. 14). These data show that centSIRT6 displays enhanced mADPr activity.

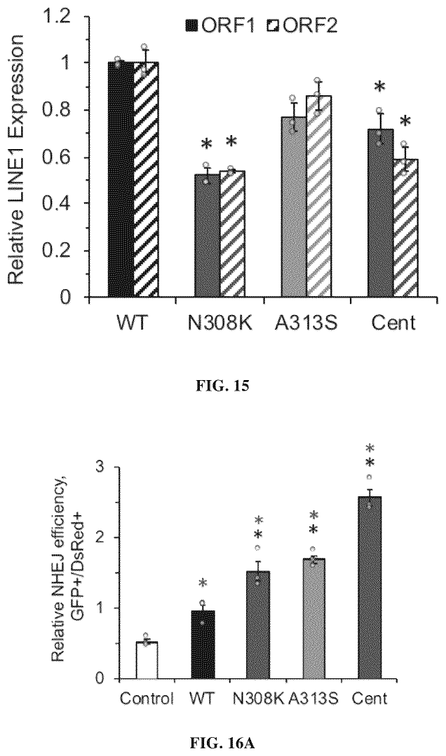

The loss of silencing of these elements contributes to age-related sterile inflammation and drives progeroid phenotypes in SIRT6 KO mice. To assess the capacity of the centSIRT6 allele to silence LINE1 transposons, the cumate-inducible fibroblasts expressing alleles of SIRT6 were used. qRT-PCR analysis of both 5′-and 3′-biased regions of an evolutionarily active family of LINEIs both showed that the centSIRT6 allele enhances LINE1 suppression compared to the wild type SIRT6, as did the N308K allele (FIG. 15). The A313S allele showed a slight trend towards stronger suppression, but did not show a significant improvement over the wild type SIRT6 when assessed for the 3′ bias. These results show that centSIRT6 is more efficient at silencing LINE1 elements, which are functionally implicated in longevity.

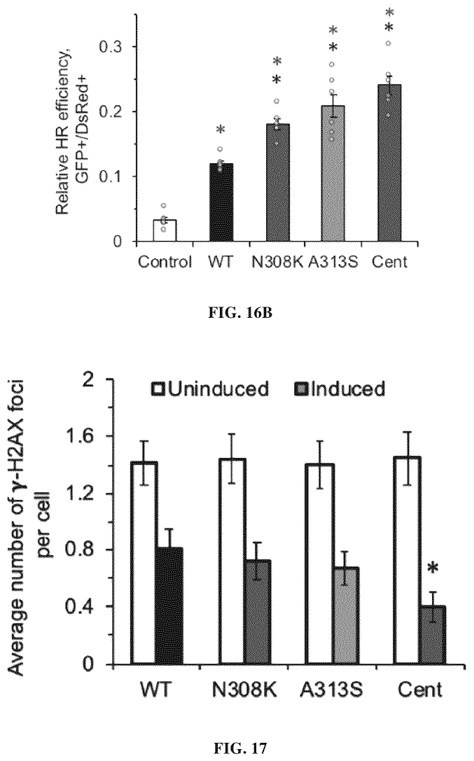

To quantify the differences between the SIRT6 alleles at promoting DSB repair, in vivo GFP-based assays that measure nonhomologous end joining (NHEJ) and homologous recombination (HR) repair of a chromosomal DSB induced by I-Scel enzyme were employed. Different alleles of SIRT6 were transiently expressed in the NHEJ and HR reporter telomerase-immortalized human foreskin fibroblast cell lines. It was found that equivalent amounts of the centSIRT6 stimulated NHEJ and HR 2.5-fold and 2-fold greater respectively, than the wild type SIRT6 (FIG. 16A-16B). Similarly, it was observed in the cumate SIRT6 fibroblasts expressing centSIRT6 that basal levels of γH2AX foci were reduced by 50% relative to those observed in either single alleles or the wild type SIRT6 (FIG. 17). Taken together, these data indicate that the centSIRT6 allele elicits enhanced DNA repair activity.

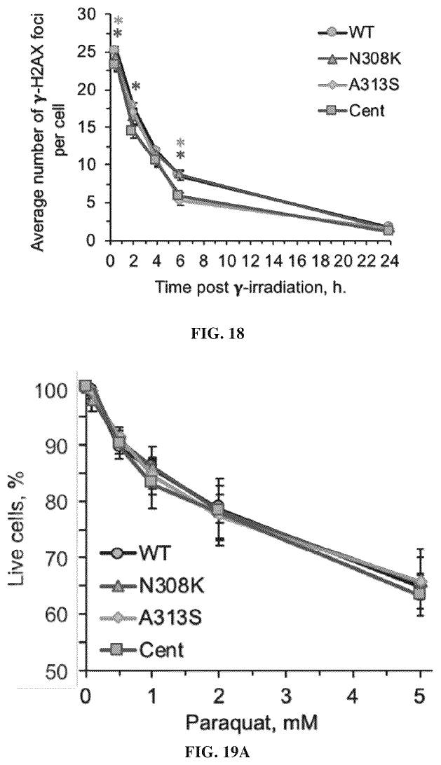

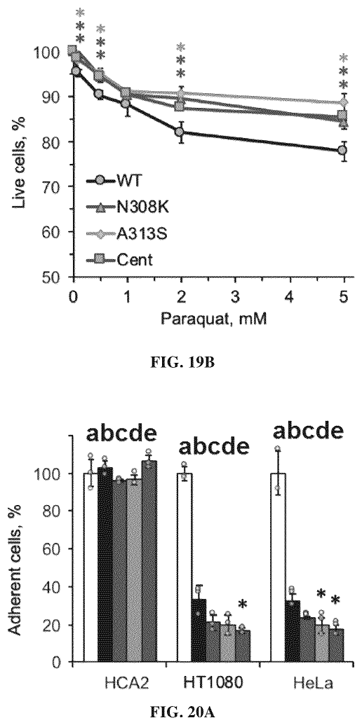

It was next compared whether different SIRT6 alleles have different effects upon stress. Using the cumate SIRT6 fibroblasts, the cells were exposed to γ-radiation and the resolution of DSBs was assessed over time via γH2AX immunostaining. It was observed that, while both wild type and centSIRT6-expressing cell lines had similar levels of γH2AX foci immediately after exposure, the centSIRT6-expressing cells showed an improved recovery rate (FIG. 18). Furthermore, it was found that the centSIRT6-expressing cells were more resistant to oxidative stress-induced apoptosis (FIG. 19A-19B). In contrast, in the absence of cumate induction, all cell lines showed no significant differences in survival after oxidative stress (FIG. 19A). Taken together, these results indicate that the centSIRT6 improves DSB repair and resistance to DNA damage.

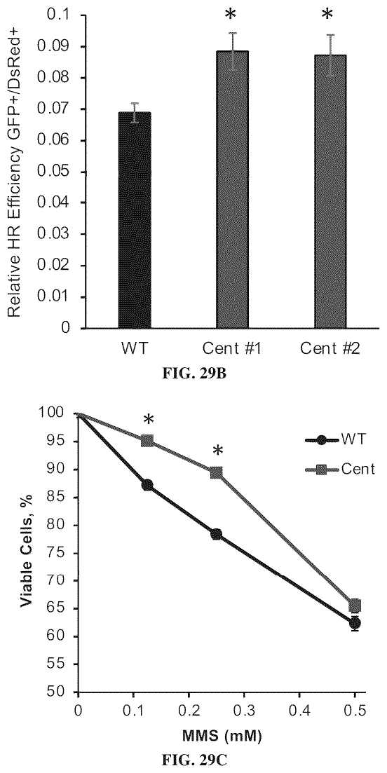

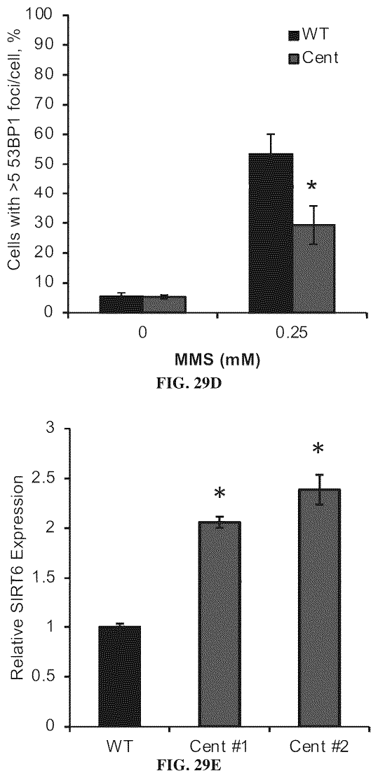

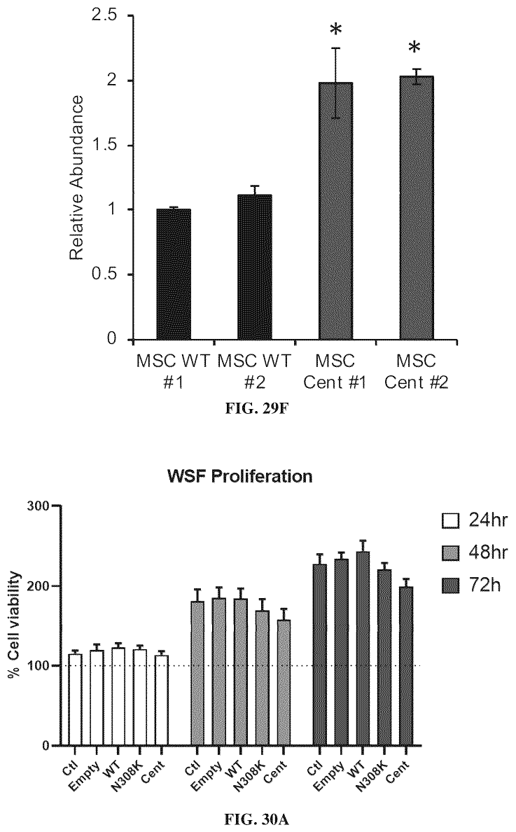

To further confirm that centSIRT6 enhances genome maintenance, a knock-in human embryonic stem cells carrying centSIRT6 allele was generated. The two centenarian mutations were introduced using CRISPR CAS9, and confirmed by sequencing. Two independent knock-in clones were generated and differentiated into MSCs. The MSCs were then transfected with linearized NHEJ and HR GFP reporters. Cells harboring centSIRT6 allele showed higher efficiency of repair (FIGS. 29A-29B). These cells were also showed increased survival and lower number of 53BP1 foci upon treatment with methyl methanesulfonate (MMS), a potent DNA-damaging agent (FIGS. 29C-29D). Interestingly, the centSIRT6 knock-in cells showed higher protein and mRNA levels of SIRT6 (FIGS. 29E-29F), suggesting that centenarian mutations increase mRNA stability, in addition to modulating SIRT6 enzymatic activities. As it was impossible to compare equal levels of SIRT6 in CRISPR knock-in cells, the majority of in vivo assays relied on cumate cells.

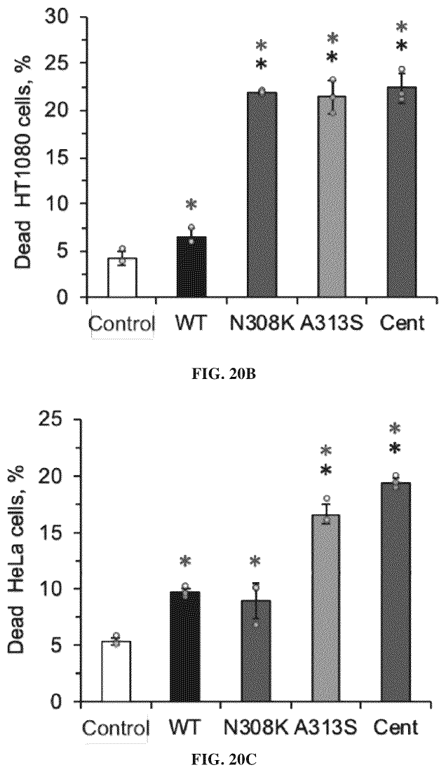

In order to assess the tumor cell killing capacity of the centSIRT6 allele, SIRT6 alleles were transfected into two common tumor cell lines, HT1080 fibrosarcoma cells and HeLa cells. The centSIRT6 showed ˜2-fold fewer adherent surviving cells in both cancer cell lines (FIG. 20A). The centSIRT6 allele triggered at least 2-fold higher levels of apoptosis than the wild type SIRT6 allele (FIG. 20B-20C). These data indicate that, centSIRT6 confers a more robust anti-tumor activity than its wild type equivalent.

Since the centSIRT6 mutations are located in the flexible C-terminus of SIRT6 and may influence protein-protein interactions, the interactomes of the centSIRT6 and the wild type alleles were compared. Antibodies against SIRT6 were used to immuno-precipitate SIRT6-interacting proteins from cumate SIRT6 fibroblasts and to analyze them by mass spectrometry with tandem-mass tags (TMT) to permit accurate quantitation. Several interacting proteins were enriched by the centSIRT6 allele (Table 7).

TABLE 7
Proteins showing stronger interaction with
centSIRT6 compared to the wild type SIRT6.

| Name | Description | Peptides | p value |
|---|---|---|---|
| RBP56 | TATA-binding protein-associated | 5 | 0.0002 |
| | factor 2N | | |
| LMNA | Prelamin-A/C | 38 | 0.0025 |
| RL35 | 60S ribosomal protein L35 | 4 | 0.0056 |
| CEBPB | CCAAT/enhancer-binding protein | 1 | 0.0119 |
| | beta | | |
| VIME | Vimentin | 40 | 0.0188 |
| NUMA1 | Nuclear mitotic apparatus protein 1 | 2 | 0.0227 |
| NOL6 | Nucleolar protein 6 | 2 | 0.0250 |
| FRG1 | Protein FRG1 | 2 | 0.0336 |
| A0A0B4J2E5 | Uncharacterized protein | 3 | 0.0354 |
| PHRF1 | PHD/RING finger domain-containing | 3 | 0.0445 |
| | protein 1 | | |
| H15 | Histone H1.5 | 11 | 0.0516 |
| H31 | Histone H3.1 | 3 | 0.0534 |
| NOL10 | Nucleolar protein 10 | 1 | 0.0581 |

Proteins were prepared from cumate-induced SIRT6 cell lines and immuno-precipitated with SIRT6 antibody; rabbit pre-immune serum was used as a control. Prior to analysis by mass spectrometry, samples were labeled with tandem-mass tags.

Most notable proteins showing stronger interaction with centSIRT6 than the wild type SIRT6 were LMNA and vimentin (VIME), which showed 38 and 40 peptides respectively. Both of these proteins are known to interact with SIRT6, and LMNA was shown to stimulate SIRT6 activity. No loss or gain of interactions was apparent in the centSIRT6 set, indicating that the effect of the novel allele could be in enhancing existing SIRT6 functions.

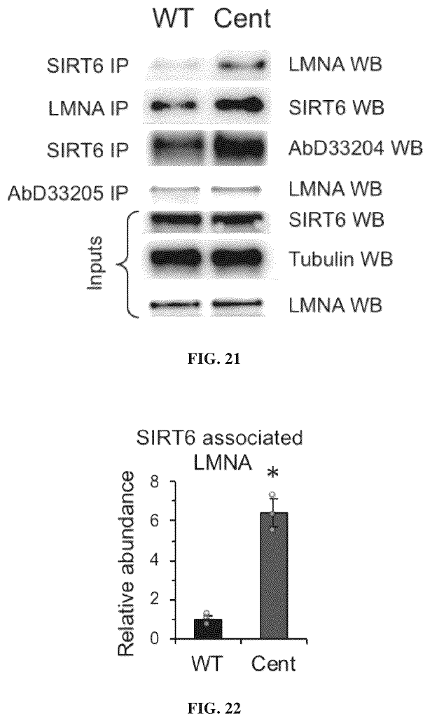

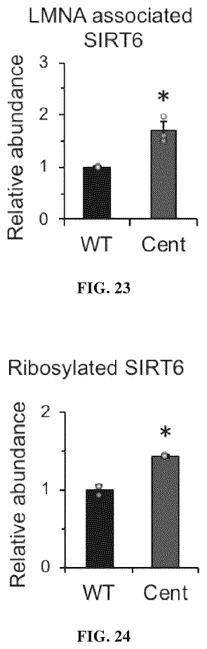

LMNA is a nuclear scaffold protein playing a central role in nuclear organization and is also implicated in ageing. Aberrant processing of LMNA results in human premature ageing syndrome, Hutchison Gilford Progeria, while LMNA SNPs have been identified in human centenarians. Fibroblasts isolated from centenarians were also shown to have increased levels of pre-LMNA, suggesting that modulated functions of LMNA are associated with both premature ageing and exceptional longevity. To confirm the mass spec data on SIRT6 and LMNA interactions, co-IP were performed with SIRT6 antibodies followed by Western blot with LMNA antibodies using cumate SIRT6 fibroblasts. It was found that centSIRT6, indeed, associates more strongly with LMNA than the wild type SIRT6 allele (FIG. 21 and FIG. 22) and the effect was reciprocal as assessed by co-IP with LMNA antibodies followed by Western blot with SIRT6 antibodies (FIG. 21 and FIG. 23). Taken together these results demonstrate that centSIRT6 interacts more strongly with LMNA than its wild type counterpart.

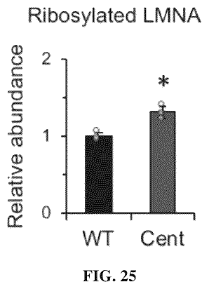

Given the enhanced mADPr activity of the centSIRT6 allele, it was next assessed the ribosylation status of LMNA using the mADPr antibodies. SIRT6 IPs performed on the cumate SIRT6 fibroblasts, using the mADPr specific antibody, showed that centSIRT6 was more ribosylated in vivo (FIG. 24); as demonstrated in vitro with the purified SIRT6 proteins (FIG. 13). IP with mADPr specific antibody and subsequent Western blot with an anti-LMNA antibody revealed increased ribosylation of LMNA in the presence of the centSIRT6 allele (FIG. 25). LMNA has been reported to be ADP ribosylated in vivo on several residues.

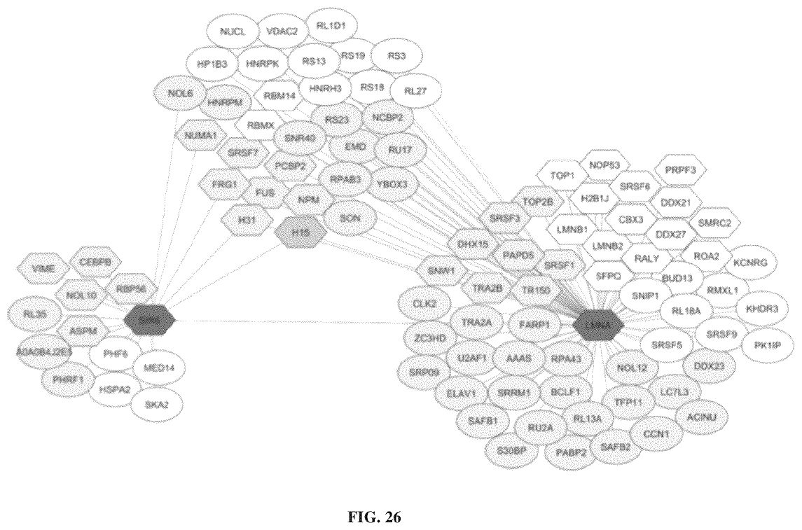

It was hypothesized that the centSIRT6 allele may influence the interaction of LMNA with other proteins. To test this, LMNA from the cumate fibroblasts expressing either the wild type SIRT6 or centSIRT6 alleles were immuno-precipitated and quantitative protein interaction analysis using TMT labeling followed by mass spectrometry were performed. While many proteins showed similar interaction with LMNA, regardless of the SIRT6 allele present, a large group of proteins showed enhanced interaction with LMNA in the presence of centSIRT6 (FIG. 26). LMNA-interaction partners enhanced by sentSIRT6 included ELAVLI, FUS, PCBP2, SAFB, SRSFI, SRSF3, TFIP11, THRAP3, BCLAFI, and U2AFBP all proteins that facilitate the coordination of the DNA damage response with RNA processing. Many of the proteins identified in SIRT6 and LMNA IPs are ADP-ribosylated (shown as hexagons in FIG. 26), suggesting that ADP-ribosylation by SIRT6 or SIRT6-activated PARP1 may play a role in mediating these interactions. Taken together, these data show that centSIRT6 has enhanced capability to promote DNA repair and suppress LINE1 elements, which is likely mediated by its enhanced mADPr activity and stronger interaction with LMNA.

### DISCUSSION

The discovery of beneficial SIRT6 mutations associated with enhanced mADPr activity in a centenarian cohort suggests that increased SIRT6 activity benefits human longevity. While increased SIRT6 activity correlates with longer lifespan in model organisms, it has not been clear which of SIRT6's enzymatic activities are central to longevity. Interestingly, loss of SIRT6 deacetylation activity in humans or SIRT6 knockout in monkeys lead to severe developmental phenotypes rather than premature ageing, suggesting that, at least in primates, deacetylation activity of SIRT6 is required for development but not necessarily for adult longevity. The change in the balance of deacetylation and mADPr enzymatic activities leads to enhanced function of SIRT6 in DNA repair, LINE1 suppression and cancer cell killing, which require mADPr activity. The centSIRT6 variant also shows enhanced binding to LMNA which may further promote its function in DNA repair and chromatin organization via direct stimulation of SIRT6 enzymatic activity, and by coordinating interactions of LMNA with other components of the LMNA complex.

### Example 2

### Materials and Methods

### Cell Culture

3T3-L1, LX2, HepG2, Hep3B (all purchased from American Type Culture Collection-ATCC, Manassas) and WSF (purchased from The Coriell Institute for Medical Research, NJ) cells were grown in basal media cosnsisting of high glucose (4.5 g/l) DMEM (LM-D1108/500, Biosera) supplemented with 10% bovine fetal calf serum (F9665, Sigma-Aldrich), 15 mM hepes buffer (L0180, Biowest), GlutaMAX™ Supplement (100x, 35050-038, Gibco), 100 U/ml penicillin and 100 mg/ml streptomycin solution (L0022, Biowest), 100 nM sodium pyruvate (11360, Gibco) and non-essential amino acids (NEAA 11140, 100x, Gibco) in 5% CO2/humidified atmosphere at 37° C. The medium was routinely changed every 2 days and the cells were sub-cultured using TrypLE Express (1x, Gibco) when reaching 90% confluence.

IHH cells were purchased from American Type Culture Collection (ATCC, Manassas, VA) and cultured in Williams E medium (12551032, Gibco) containing 2 g/l glucose and supplemented with 10% fetal calf serum, 100 U/ml penicillin and 100 μg/ml streptomycin solution, 20 mU/ml insulin and 50 nM dexamethasone in 5% CO2/humidified atmosphere at 37° C. The cell culture medium was changed every 2 days and the cells were sub-cultured using TrypLE Express when reaching 90% confluence.

For the purpose of microscopy analyses, all appropriate cell lines were seeded on glass coverslips coated with 0.2% gelatin with no further modification of standard cultivation protocols. For the evaluation of the effect of SIRT6 variants and their overexpression on tissue fibrosis, appropriate cell lines were treated or not with recombinant TGF-β1 protein (100-21C, Peprotech, US), as a potent inductor of fibrotic process, at concentration of 10 and 20 ng/ml.

### Lentivirus Infection and Establishing SIRT6 Cell Lines

The cells, after reaching confluence, were split and seeded into 24-well plate with growth surface area of 2 cm², where 50×10⁴ cells per well were seeded. Immediately after seeding, cells were infected with Lentivirus containing SIRT6 constructs: LV2-EMPTY-IresKat2S, LV2-SIRT6(wt)-IresKat2S, LV2-SIRT6(N308K)-IresKat2S and LV2-SIRT6(centSIRT6)-IresKat2S, in basal DMEM media supplemented with 4 μg/mL polybrene transfection reagent (TR-1003, Sigma-Aldrich) with MOI of 1-2. Cells were cultivated with Lentivaral constructs for the period of 24 hours, then the fresh medium was added and cells were cultivated for another 48 hours. Then the cells were treated with selection media comprising of basal DMEM media with 500 μg/mL of hygromycin (H3274, Merck) for the period of 5 days (every other day the media was changed for the fresh one). After selection, remaining cells were expanded and checked for the presence of fluorescent Kat2S signal by fluorescence microscope ex/em 588/635. SIRT6 overexpression was confirmed by western-blotting.

### 3T3-L1 Differentiation to Adipocytes

Differentiation of 3T3-L1 adipocytes was induced 2 days post-seeding at 100% confluence by incubating cells for the period of 72 hours in differentiation media consisting of standard basal high-glucose DMEM media additionally supplemented with 500 μM IBMX (15879, Sigma-Aldrich), 1 μM rosiglitazone (15879, Sigma-Aldrich), 250 nM dexamethasone (D4902, Sigma-Aldrich), 10 nM 3,3′,5′-Triiodo-L-thyronine (T6397, Sigma-Aldrich), 17 μM D-Pantothenic acid (21210, Sigma-Aldrich), 10 μg/ml human transferrin (T8158, Sigma-Aldrich) and 1 μg/ml insulin (19278, Sigma-Aldrich). Then the cells are kept in the maintenance media consisting of basal high-glucose DMEM media supplemented only with 25 nM dexamethasone and 0.1 μg/ml insulin for the period of at least 4 days. Adipogenic differentiation and/or accumulation of intracellular lipids was evaluated using fluorescence labeling by BODIPY dye (1 μg/ml) for 30 minutes (ex/em 493/503) and DAPI counterstain 1 μg/mL for 30 min. Fluorescence was then captured by Agilent BioTek FLx800 Microplate Fluorescence Reader equipped with appropriate excitation/emission filters.

### Cell Viability Assay

Cells were seeded into 96-well plates at 2000 viable cells per well and after 24, 48 and 72 hours, the culture media was removed and replace with medium containing Hoechst dyes at 1 μg/mL for nuclear staining. After 20 minutes of incubation at 37° C., the cells were twice washed with PBS and the fluorescence signal was measured with Agilent BioTek FLx800 Microplate Fluorescence Reader at ex/em 355/488 nm. The ratio between the signal at different time points and TO was used as relative percentage of viable cells.

### Gene Expression Measurement

Genomic RNA was isolated by column separation techniques using an RNeasy mini Kit (74106, Qiagen, Germany), according to manufacturer's instructions. At least 4 biological replicates were prepared for each treatment group. After quantification of isolated total RNA on NanoDrop 1000 spectrophotometer (ThermoFisher Scientific), 1 μg of total isolated RNA was used to prepare cDNA using a High-Capacity cDNA Reverse Transcription Kit (4368814, ThermoFisher Scientific). Real Time-PCR was performed in at least two technical replicates using a StepOnePlus™ Real-Time PCR System (Applied Biosystems) and SYBR™ Select Master Mix (4472908, ThermoFisher Scientific). The PCR reaction was held in 10 μl volume and 250 ng cDNA per well was used as the input quantity. The primer sequences used in this study are listed in Table 8.

TABLE 8
Primer sequences used in the study

| Gene | Sequence (5′-3′) | | SEQ ID NO: |
|---|---|---|---|
| αSMA | F | AAAAGACAGCTACGTGGGTGA | 13 |
| | R | GCCATGTTCTATCGGGTACTTC | 14 |
| COL1A1 | F | GTGCGATGACGTGATCTGTGA | 15 |
| | R | CGGTGGTTTCTTGGTCGGT | 16 |
| TIMP1 | F | ACCACCTTATACCAGCGTTATGA | 17 |
| | R | GGTGTAGACGAACCGGATGTC | 18 |
| VIMENTIN | F | AGTCCACTGAGTACCGGAGAC | 19 |
| | R | CATTTCACGCATCTGGCGTTC | 20 |
| MMP2 | F | TACAGGATCATTGGCTACACACC | 21 |
| | R | GGTCACATCGCTCCAGACT | 22 |
| GAPDH | F | GGTGCGTGCCCAGTTGA | 23 |
| | R | TACTTTCTCCCCGCTTTTT | 24 |
| Actinβ | F | CATGTACGTTGCTATCCAGGC | 25 |
| | R | CTCCTTAATGTCACGCACGAT | 26 |

### Telomere Length

Quantitative RT-PCR was used to determine changes in average telomere length of LV transfected WSF cells and untreated WSF based on ScienCell's Relative Human Telomere Length Quantification qPCR Assay Kit (#8909). The kit directly compares the average telomere length of the samples. The telomere primer set recognizes and amplifies telomere sequences. The single copy reference (SCR) primer set recognizes and amplifies a 100 bp-long region on human chromosome 17, and serves as reference for data normalization. The carefully designed primers ensure: (i) high efficiency for trustworthy quantification; and (ii) no non-specific amplification. Each primer set has been validated by qPCR with melt curve analysis and gel electrophoresis for amplification specificity and by template serial dilution for amplification efficiency. Genomic DNA (gDNA) was isolated from using the Qiagen DNA Isolation Kit (Qiagen, Hilden, Germany). Each PCR reaction contained genomic DNA sample (0.01 μg/μL), telomere primer, 2× qPCR master mix, and nuclease-free water. Primer-probe real-time PCR was performed using StepOnePlus™ Real-Time PCR System (Applied Biosystems). All reactions were performed in duplicate, and template controls were included in each run. Amplification was performed under the following conditions: denaturation for 10 min at 95° C. followed by 32 cycles of denaturation for 20 sec at 95° C., annealing for 20 sec at 52° C., and extension for 45 sec at 72° C. The average telomere length was calculated by following the manufacturer's instructions.

### Quantification of Telomerase Activity

Relative telomerase activity of LV WSF transfected cell lines and normal WSF cells was assessed by qPCR in StepOnePlus™ Real-Time PCR System (Applied Biosystems) using SYBR® Green assay kits (ScienCell's Telomerase Activity Quantification qPCR Assay Kit [TAQ], Carlsbad, CA, USA) following the recommendations of the manufacturers. Three million cells were harvested for each sample. For each experiment, two controls were used: a negative telomerase and positive cell lysates (Cat #8928e). PCR was performed in a final volume of 20 μL using 1 μL of post-telomerase reaction sample, 2 μL of TPS, 10 μL of 2× qPCR FastStart Essential Green Master Mix (Roche Diagnostics International) and 7 μL of nuclease-free water. The PCR conditions were as follows: 95° C. for 10 min followed by 36 cycles of: 95° C. for 20 sec, 52° C. for 20 sec and 72° C. for 45 sec. All reactions were run in three replicates. Data analysis was conducted according to manufacturer's instructions.

### Immunoblotting Analyses

Cells were harvested from cultivation plates using TrypLE Express, washed with 1×PBS and centrifuged at 300 g. After discarding supernatant, obtained pellet was resuspended 1× Rippa lysis buffer (20-188, Millipore, USA) supplemented with Halt™ Protease and Phosphatase Inhibitor Cocktail (100X, Thermo Fisher) and lysed on ice (4° C.) for 30 minutes with vigorous vertexing every 10 min. After spinning at 10 000 g for 10 min, supernatant was moved to new vial and concentration of protein was measured by Pierce™ BCA Protein Assay Kit (23225, Thermo Fisher) according to manufacturer's instruction. Equal amount of protein sample (at least 20 μg) was mixed with 1× Laemmli Sample buffer (1610747, 4x, Bio-Rad) and after boiling at 95° C. for 5 min and cooling on ice, equal volume of proteins (40 μl) was loaded on 10% Mini-PROTEAN® TGX Stain-Free™ Protein Gels (4568034, Bio-Rad) and separated by electrophoresis running at 120 volts for 45 minutes. Protein transfer was performed on PVDF membranes using Trans-Blot Turbo RTA Mini 0.45 μm LF PVDF Transfer Kit (1704274, Bio-Rad) and Bio-Rad Trans-Blot Turbo Transfer System at 1.3 A and 25 V for 10 min. Membranes were then blocked with 5% bovine serum albumin (BSA, P6154, BioWest) dissolved in TBST buffer (20 mM Tris-HCl, pH 7.6, 140 mM NaCl, 0.1% Tween 20) for at least 30 minutes and incubated with the specific primary antibodies (see below) diluted in TBST blocking solution, at appropriate dilutions. Following three washes in TBST buffer, membranes were incubated with a secondary antibody conjugated with horseradish peroxidase diluted in TBST blocking buffer. After three further washes with TBST, proteins were revealed by Clarity Western ECL Substrate (1705061, Bio-Rad) and the signal detected on Bio-Rad ChemiDoc XRS+ imaging systems. For quantitative measurement, the scanned membranes were analyzed using the Image Lab™ Software (Bio-Rad).

Antibodies used in this study: Cell Signaling Technology (MA, USA)-rabbit anti-Akt (1:1000), rabbit anti-Phospho-Akt (Ser473) (1:1000), rabbit anti Vimentin (1:1000), rabbit anti Histone H3 (D1H2, 1:1000), Abcam (UK)-mouse anti-β-actin HRP conjugated antibody (AC-15; 1:2000), mouse anti-GAPDH monoclonal HRP conjugated antibody (1:2000), rabbit anti Collagen I (1:1000), rabbit anti aSMA (1:1000), rabbit anti SIRT6 antibody (1:1000, EPR18463), ThermoFischer Scientific (CA, USA)-mouse IgG1 GAPDH monoclonal HRP conjugated antibody (1:2000), secondary goat Anti-rabbit IgG HRP-linked (1:2000);

### 3D Spheroid Culture

For the generation of the cell spheroids, cells were seeded into BIOFLOAT™ 96-well round bottom ultra-low attachment plates for cell culture at 10000 viable cells per well. Each IHH LV transfect cell line (CTL/EMPTY, WT, N308K and centSIRT6) was co-cultured with either normal LX2 or overexpressing centSirt6 LX2 cells with 20:1 ratio, to reproduce the physiological proportion in the liver parenchyma, where hepatocytes are major cell type with only ≈5% hepatic stellate cells. The spheroids were grown in basal high glucose DMEM media supplemented as described above. The plates were incubated for 3 days at 37° C. in a humidified atmosphere at 5% CO2, after which spheroids were treated or not with free fatty acid solution (FFA, L9655, Sigma-Aldrich) and kept in cultivation for 48 hours. Spheroids were then collected, washed twice with PBS, fixed in 4% paraformaldehyde+Eosin solution for 20 minutes, incubated in 10% sucrose for 20 minutes and incorporated into tissue freezing media (14020108926, Leica Biosystems, USA) for cutting and subsequent immuno-histological analysis.

### Histological Immuno-Staining

Samples of embedded and snap frozen spheroids in tissue freezing media were cut to 7 μm at −20° C. with a cryotome (Leica Microsystems) and stored at −80° C. for further use. To evaluate the effect of SIRT6 variants and their overexpression on liver tissue fibrosis, spheroid histological sections were immunolabeled to detect Collagen 1A. Slides were washed once in 1xPBS to dissolve tissue freezing media and blocked in 1xPBS supplemented with 0.2% Tween-20 and 5% BSA. Primary antibody rabbit anti-Collagen I (1:500, ab34710, Abcam) was diluted in DAKO antibody diluent (S202230-2, Agilent technologies) and incubated overnight in humid chamber at lab temperature. After three consecutive washes with 1xPBS, mix of secondary antibody (1:500) donkey anti-rabbit IgG coupled with Alexa Fluor™ 647 was applied and incubated for at least 1 h. After washing three times with 1xPBS, slides were counterstained with DAPI (1 μg/ml) solution for 15 minutes and mounted in Mowiol hardening media. After hardening (overnigth at 4° C.), images were captured using an Axio scan Z.1 (ZEISS) equipped with a Hamamatsu ORCA-Flash 4.0 camera and ImageJ software (NIH, USA) analysis program was used to evaluate all immunofluorescence images. Fibrosis, i.e., collagene 1A abundance in spheroid samples, was evaluated as the % of the total spheroid area delineated by DAPI fluorescence at 100× magnification, when at least 5 spheroids per each condition/cell line were used in three consecutive and independent experiments.

### Results

SIRT6 wild type or SIRT6 carrying one or two mutations associated with human exceptional longevity (SIRT6 N308K, centSIRT6) were overexpressed in a variety of cell lines, and assessed how this affected their specific cellular functions.

### Werner Syndrome Model

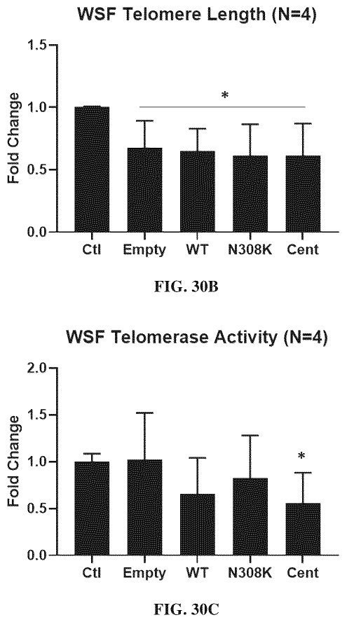

In human Werner Syndrome immortalized fibroblasts, SIRT6 WT/SIRT6 N308K/centSIRT6 did not affect cell viability, proliferation or telomere length (FIG. 30A-30B). However, centSIRT6 decreased telomerase activity (FIG. 30C).

Although it may seem counter-beneficial, Werner Syndrome immortalized fibroblasts are poorly characterized immortalized lines that might have carcinogenic properties. Therefore, without being bound by any theory, and in the event that this is the case, decreasing telomerase activity would be a beneficial effect.

### Human Hepatocellular Carcinoma Model

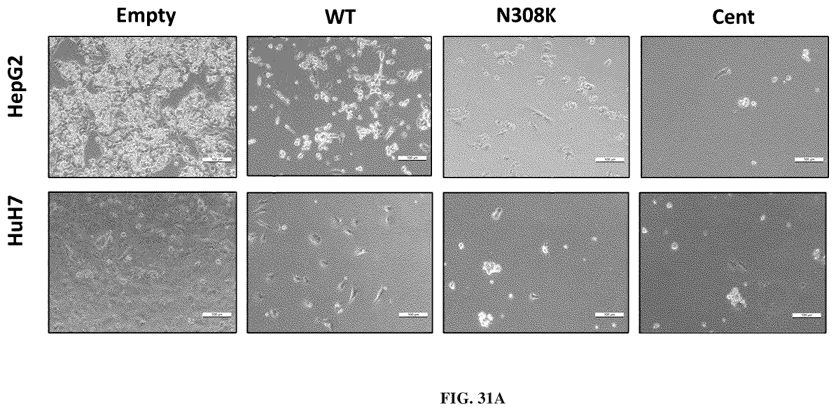

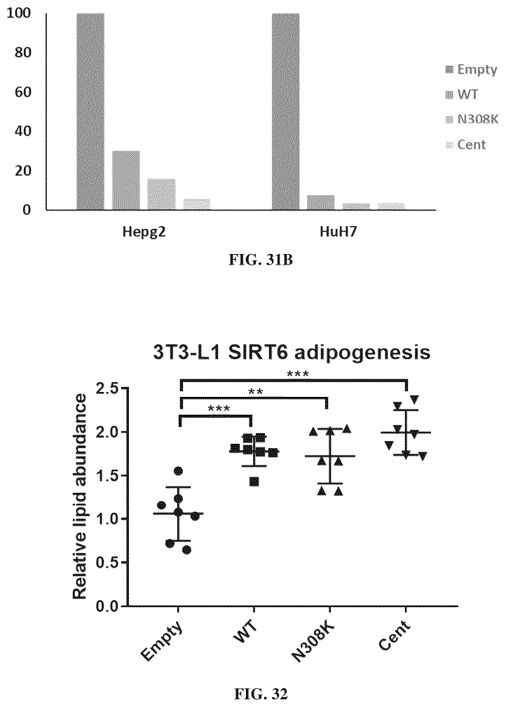

In human hepatocellular carcinoma (HCC) cells, SIRT6 WT/SIRT6 N308K/centSIRT6 overexpression was lethal, with a larger effect observed for SIRT6 N308K/centSIRT6 (FIG. 31A-31B). Therefore, mutated SIRT6 may be useful for treating cancer.

### Adipogenic Differentiation

It has been reported that SIRT6 deficiency results in hypoglycemia and impairment of energy homeostasis, suggesting that SIRT6 could play an important role in regulating adipocyte differentiation. Aged adipocytes become hypertrophic, in particular under a high fat diet intake, becoming insulin resistant and producing a wide pattern of pro-inflammatory cytokines, contributing to the increase of circulating triglycerides and free fatty acids, which together affects other organs and results in the comorbidities typically associated with obesity. On the other hand, the increase of adipogenesis allows the formation of new adipocytes that are more sensitive to insulin, showing increased capacity of fatty storage and enhanced levels of adipokines, that contribute to metabolic homeostasis and lowering of inflammation levels.

Here, the results demonstrate that SIRT6 plays a role in the increase of adipogenic differentiation (FIG. 32), consistent with the role of this protein in energy homeostasis. The SIRT6 mutants (SIRT6 N308K and centSIRT6) retain this property, which is of interest for the treatment of ageing and ageing-related diseases.

### Hepatic Stellate Cells Model

α-SMA expression is considered a reliable marker of hepatic stellate cell (HSC) activation and a key biomarker for liver fibrosis. In the liver, HSCs play a central role in physiological tissue repair as well as in the development of fibrosis, transdifferentiating from “quiescent” HSC to a myofibroblastic phenotype in response to Transforming Growth Factor beta (TGF-β), inflammatory processes and ROS.

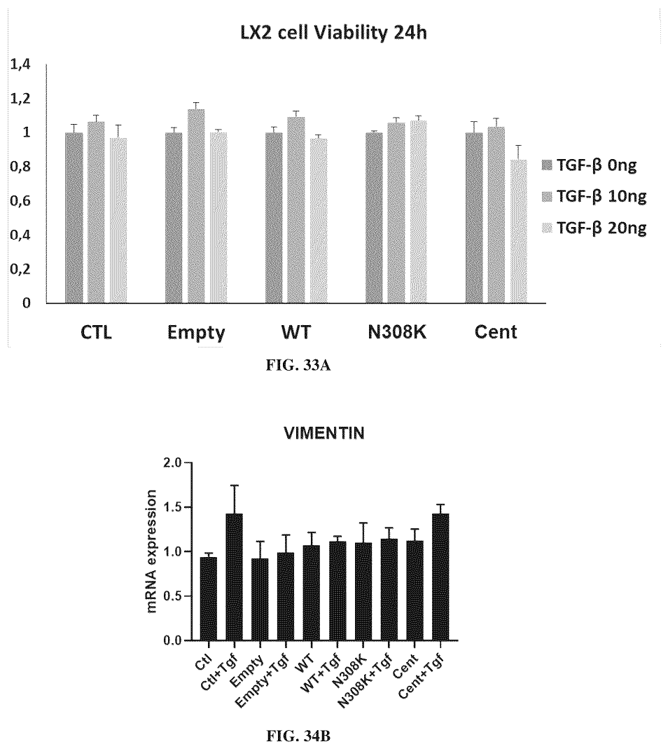

SIRT6 WT/SIRT6 N308K/centSIRT6 overexpression did not affect cell viability or proliferation (FIG. 33A). Upon LX2 activation with TGFbeta, there was a blockage of the activation of some fibrogenic markers (vimentin, TIMP1) by lentiviral infection, irrespective of the genotype (FIG. 33B-33C). Collagen mRNA expression was increased by TGFbeta, with no effect of SIRT6 WT/SIRT6 N308K/centSIRT6 overexpression (FIG. 33D). However, centSIRT6 significantly decreased the mRNA expression of aSMA, a major fibrogenic marker (FIG. 33E).

The lower gene expression levels of α-SMA in centSIRT6 LX2 compared to control suggests a potential anti-fibrotic effect of centSIRT6 in preventing liver fibrosis and/or promoting its resolution.

### Hepatic Spheroids Model

Together with α-SMA, Collagen (COL1A1) represent one of the key fibrosis markers. Its production in liver is increased mainly by activated hepatic stellate cells.

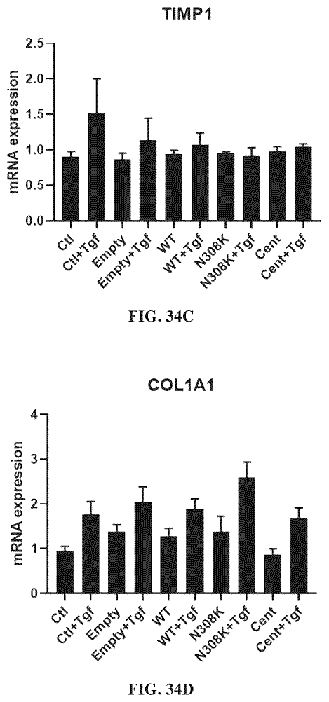

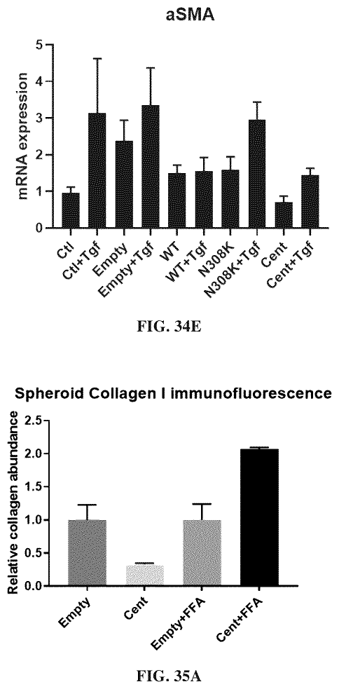

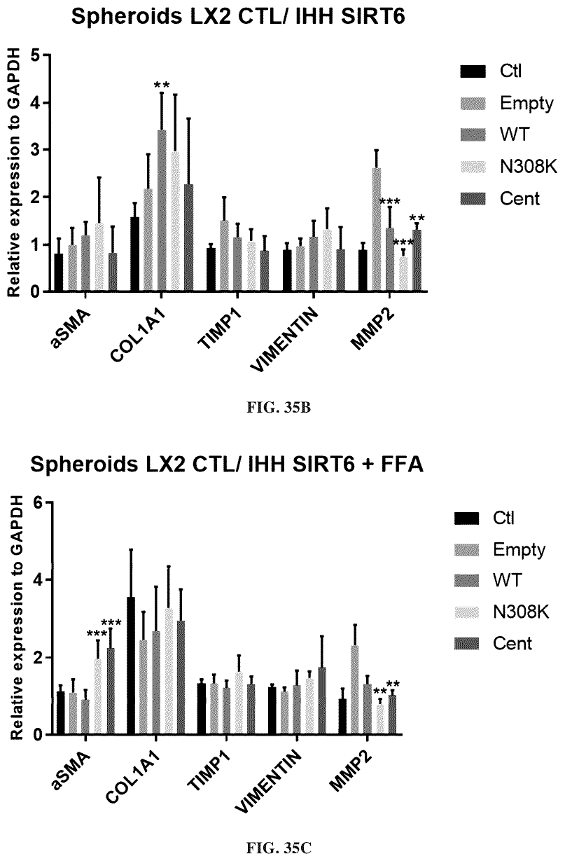

Spheroids formed from IHH cells and with 5% the total cell mass of LX2 (human hepatic stellate cells) were generated. Spheroids were treated or not with free fatty acids (FFA), and assessed collagen production by immunofluorescence (FIG. 34A).

In IHH/LX2 spheroids with IHH transfected with centSIRT6, a decrease of internal collagen content compared to control (Empty group) was observed, suggesting that at the basal levels, in absence of stressor factors, IHH centSIRT6 inhibit the collagen production (FIG. 34B).

It is known that exposure to free fatty acids (FFAs) promotes oxidant stress and indirect activates LX2 increasing collagen biosynthesis. Surprisingly, in presence of FFAs, increased levels of internal collagen in IHH centSIRT6 spheroids compared to control were observed (FIG. 34C). Increased levels of free fatty acids together with enhanced beta-oxidation induced by centSIRT6 overexpression may have provided more ATP necessary for the acquisition of the myofibroblastic phenotype of fibroblasts, overcoming the inhibitory effect on collagen production exerted by centSIRT6 overexpression observed on the basal level.

SEQUENCES USED HEREIN

SEQ ID NO: 1 - Wild type SIRT6 amino acid sequence
MSVNYAAGLSPYADKGKCGLPEIFDPPEELERKVWELARLVWQSSSVV
FHTGAGISTASGIPDFRGPHGVWTMEERGLAPKFDTTFESARPTQTHMALVQLE
RVGLLRFLVSQNVDGLHVRSGFPRDKLAELHGNMFVEECAKCKTQYVRDTVV
GTMGLKATGRLCTVAKARGLRACRGELRDTILDWEDSLPDRDLALADEASRN
ADLSITLGTSLQIRPSGNLPLATKRRGGRLVIVNLQPTKHDRHADLRIHGYVDEV
MTRLMKHLGLEIPAWDGPRVLERALPPLPRPPTPKLEPKEESPTRINGSIPAGPK
QEPCAQHNGSEPASPKRERPTSPAPHRPPKRVKAKAVPS
SEQ ID NO: 2 - SIRT6 N308K variant amino acid sequence
MSVNYAAGLSPYADKGKCGLPEIFDPPEELERKVWELARLVWQSSSVV
FHTGAGISTASGIPDFRGPHGVWTMEERGLAPKFDTTFESARPTQTHMALVQLE
RVGLLRFLVSQNVDGLHVRSGFPRDKLAELHGNMFVEECAKCKTQYVRDTVV
GTMGLKATGRLCTVAKARGLRACRGELRDTILDWEDSLPDRDLALADEASRN
ADLSITLGTSLQIRPSGNLPLATKRRGGRLVIVNLQPTKHDRHADLRIHGYVDEV
MTRLMKHLGLEIPAWDGPRVLERALPPLPRPPTPKLEPKEESPTRIKGSIPAGPK
QEPCAQHNGSEPASPKRERPTSPAPHRPPKRVKAKAVPS
SEQ ID NO: 3 - SIRT6 A313S variant amino acid sequence
MSVNYAAGLSPYADKGKCGLPEIFDPPEELERKVWELARLVWQSSSVV
FHTGAGISTASGIPDFRGPHGVWTMEERGLAPKFDTTFESARPTQTHMALVQLE
RVGLLRFLVSQNVDGLHVRSGFPRDKLAELHGNMFVEECAKCKTQYVRDTVV
GTMGLKATGRLCTVAKARGLRACRGELRDTILDWEDSLPDRDLALADEASRN
ADLSITLGTSLQIRPSGNLPLATKRRGGRLVIVNLQPTKHDRHADLRIHGYVDEV
MTRLMKHLGLEIPAWDGPRVLERALPPLPRPPTPKLEPKEESPTRINGSIPSGPKQ
EPCAQHNGSEPASPKRERPTSPAPHRPPKRVKAKAVPS
SEQ ID NO: 4 - SIRT6 N308K A313S variant (centSIRT6) amino acid sequence
MSVNYAAGLSPYADKGKCGLPEIFDPPEELERKVWELARLVWQSSSVV
FHTGAGISTASGIPDFRGPHGVWTMEERGLAPKFDTTFESARPTQTHMALVQLE
RVGLLRFLVSQNVDGLHVRSGFPRDKLAELHGNMFVEECAKCKTQYVRDTVV
GTMGLKATGRLCTVAKARGLRACRGELRDTILDWEDSLPDRDLALADEASRN
ADLSITLGTSLQIRPSGNLPLATKRRGGRLVIVNLQPTKHDRHADLRIHGYVDEV
MTRLMKHLGLEIPAWDGPRVLERALPPLPRPPTPKLEPKEESPTRIKGSIPSGPKQ
EPCAQHNGSEPASPKRERPTSPAPHRPPKRVKAKAVPS
SEQ ID NO: 5 - Wild type SIRT6 nucleic acid sequence
atgtcggtgaattacgcggcggggctgtcgccgtacgcggacaagggcaagtgcggcctcccggagatcttcg
accccccggaggagctggagcggaaggtgtgggaactggcgaggctggtctggcagtcttccagtgtggtgttccacacgg
gtgccggcatcagcactgcctctggcatccccgacttcaggggtccccacggagtctggaccatggaggagcgaggtctgg
cccccaagttcgacaccacctttgagagcgcgcggcccacgcagacccacatggcgctggtgcagctggagcgcgtgggc
ctcctccgcttcctggtcagccagaacgtggacgggctccatgtgcgctcaggcttccccagggacaaactggcagagctcc
acgggaacatgtttgtggaagaatgtgccaagtgtaagacgcagtacgtccgagacacagtcgtgggcaccatgggcctgaa
ggccacgggccggctctgcaccgtggctaaggcaagggggctgcgagcctgcaggggagagctgagggacaccatccta
gactgggaggactccctgcccgaccgggacctggcactcgccgatgaggccagcaggaacgccgacctgtccatcacgct
gggtacatcgctgcagatccggcccagcgggaacctgccgctggctaccaagcgccggggaggccgcctggtcatcgtca
acctgcagcccaccaagcacgaccgccatgctgacctccgcatccatggctacgttgacgaggtcatgacccggctcatgaa
gcacctggggctggagatccccgcctgggacggcccccgtgtgctggagagggcgctgccacccctgccccgcccgccc
acccccaagctggagcccaaggaggaatctcccacccggatcaacggctctatccccgccggccccaagcaggagccctg
cgcccagcacaacggctcagagcccgccagccccaaacgggageggcccaccagccctgccccccacagaccccccaa
aagggtgaaggccaaggcggtccccagctga
SEQ ID NO: 6 - SIRT6 N308K variant nucleic acid sequence
atgtcggtgaattacgcggcggggctgtcgccgtacgcggacaagggcaagtgcggcctcccggagatcttcg
accccccggaggagctggagcggaaggtgtgggaactggcgaggctggtctggcagtcttccagtgtggtgttccacacgg
gtgccggcatcagcactgcctctggcatccccgacttcaggggtccccacggagtctggaccatggaggagcgaggtctgg
cccccaagttcgacaccacctttgagagcgcgcggcccacgcagacccacatggcgctggtgcagctggagcgcgtgggc
ctcctccgcttcctggtcagccagaacgtggacgggctccatgtgcgctcaggcttccccagggacaaactggcagagctcc
acgggaacatgtttgtggaagaatgtgccaagtgtaagacgcagtacgtccgagacacagtcgtgggcaccatgggcctgaa
ggccacgggccggctctgcaccgtggctaaggcaagggggctgcgagcctgcaggggagagctgagggacaccatccta
gactgggaggactccctgcccgaccgggacctggcactcgccgatgaggccagcaggaacgccgacctgtccatcacgct
gggtacatcgctgcagatccggcccagcgggaacctgccgctggctaccaagcgccggggaggccgcctggtcatcgtca
acctgcagcccaccaagcacgaccgccatgctgacctccgcatccatggctacgttgacgaggtcatgacccggctcatgaa
gcacctggggctggagatccccgcctgggacggcccccgtgtgctggagagggcgctgccacccctgccccgcccgccc
acccccaagctggagcccaaggaggaatctcccacccggatcaagggctctatccccgccggccccaagcaggagccctg
cgcccagcacaacggctcagagcccgccagccccaaacgggagcggcccaccagccctgccccccacagaccccccaa
aagggtgaaggccaaggcggtccccagctga
SEQ ID NO: 7 - SIRT6 A313S variant nucleic acid sequence
atgtcggtgaattacgcggcggggctgtcgccgtacgcggacaagggcaagtgcggcctcccggagatcttcg
accccccggaggagctggagcggaaggtgtgggaactggcgaggctggtctggcagtcttccagtgtggtgttccacacgg
gtgccggcatcagcactgcctctggcatccccgacttcaggggtccccacggagtctggaccatggaggagcgaggtctgg
cccccaagttcgacaccacctttgagagcgcgcggcccacgcagacccacatggcgctggtgcagctggagcgcgtgggc
ctcctccgcttcctggtcagccagaacgtggacgggctccatgtgcgctcaggcttccccagggacaaactggcagagctcc
acgggaacatgtttgtggaagaatgtgccaagtgtaagacgcagtacgtccgagacacagtcgtgggcaccatgggcctgaa
ggccacgggccggctctgcaccgtggctaaggcaagggggctgcgagcctgcaggggagagctgagggacaccatccta
gactgggaggactccctgcccgaccgggacctggcactcgccgatgaggccagcaggaacgccgacctgtccatcacgct
gggtacatcgctgcagatccggcccagcgggaacctgccgctggctaccaagcgccggggaggccgcctggtcatcgtca
acctgcagcccaccaagcacgaccgccatgctgacctccgcatccatggctacgttgacgaggtcatgacccggctcatgaa
gcacctggggctggagatccccgcctgggacggcccccgtgtgctggagagggcgctgccacccctgccccgcccgccc
acccccaagctggagcccaaggaggaatctcccacccggatcaacggctctatcccctccggccccaagcaggagccctg
cgcccagcacaacggctcagagcccgccagccccaaacgggagcggcccaccagccctgccccccacagaccccccaa
aagggtgaaggccaaggcggtccccagctga
SEQ ID NO: 8 - SIRT6 N308K A313S variant (centSIRT6) nucleic acid sequence
atgtcggtgaattacgcggcggggctgtcgccgtacgcggacaagggcaagtgcggcctcccggagatcttcg
accccccggaggagctggagcggaaggtgtgggaactggcgaggctggtctggcagtcttccagtgtggtgttccacacgg
gtgccggcatcagcactgcctctggcatccccgacttcaggggtccccacggagtctggaccatggaggagcgaggtctgg
cccccaagttcgacaccacctttgagagcgcgcggcccacgcagacccacatggcgctggtgcagctggagcgcgtgggc
ctcctccgcttcctggtcagccagaacgtggacgggctccatgtgcgctcaggcttccccagggacaaactggcagagctcc
acgggaacatgtttgtggaagaatgtgccaagtgtaagacgcagtacgtccgagacacagtcgtgggcaccatgggcctgaa
ggccacgggccggctctgcaccgtggctaaggcaagggggctgcgagcctgcaggggagagctgagggacaccatccta
gactgggaggactccctgcccgaccgggacctggcactcgccgatgaggccagcaggaacgccgacctgtccatcacgct
gggtacatcgctgcagatccggcccagcgggaacctgccgctggctaccaagcgccggggaggccgcctggtcatcgtca
acctgcagcccaccaagcacgaccgccatgctgacctccgcatccatggctacgttgacgaggtcatgacccggctcatgaa
gcacctggggctggagatccccgcctgggacggcccccgtgtgctggagagggcgctgccacccctgccccgcccgccc
acccccaagctggagcccaaggaggaatctcccacccggatcaagggctctatcccctccggccccaagcaggagccctg
cgcccagcacaacggctcagagcccgccagccccaaacgggageggcccaccagccctgccccccacagaccccccaa
aagggtgaaggccaaggcggtccccagctga
SEQ ID NO: 9 - LINE1 ORF1 Forward-primer
atggcgaaaggcaaacgtaag
SEQ ID NO: 10 - LINE1 ORF1 Reverse-primer
attttcggttgtgttggggtg
SEQ ID NO: 11 - LINE1 ORF2 Forward-primer
gcaggggttgcaatcctagtc
SEQ ID NO: 12 - LINE1 ORF2 Reverse-primer
ctgggtgctcctgtattgggt
SEQ ID NO: 13 - αSMA Forward-primer
aaaagacagctacgtgggtga
SEQ ID NO: 14 - αSMA Reverse-primer
gccatgttctatcgggtacttc
SEQ ID NO: 15 - COL1A1 Forward-primer
gtgcgatgacgtgatctgtga
SEQ ID NO: 16 - COL1A1 Reverse-primer
cggtggtttcttggtcggt
SEQ ID NO: 17 - TIMP1 Forward-primer
accaccttataccagcgttatga
SEQ ID NO: 18 - TIMP1 Reverse-primer
ggtgtagacgaaccggatgtc
SEQ ID NO: 19 - VIMENTIN Forward-primer
agtccactgagtaccggagac
SEQ ID NO: 20 - VIMENTIN Reverse-primer
catttcacgcatctggcgttc
SEQ ID NO: 21 - MMP2 Forward-primer
tacaggatcattggctacacacc
SEQ ID NO: 22 - MMP2 Reverse-primer
ggtcacatcgctccagact
SEQ ID NO: 23 - GAPDH Forward-primer
ggtgcgtgcccagttga
SEQ ID NO: 24 - GAPDH Reverse-primer
tactttctccccgcttttt
SEQ ID NO: 25 - Actinβ Forward-primer
catgtacgttgctatccaggc
SEQ ID NO: 26 - Actinβ Reverse-primer
ctccttaatgtcacgcacgat

1-15. (canceled)

16. An isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1.

17. The isolated nucleic acid molecule according to claim 16, wherein the nucleic acid molecule is of sequence selected in the group comprising or consisting of SEQ ID NO: 6, SEQ ID NO: 7 and SEQ ID NO: 8.

18. The isolated nucleic acid molecule according to claim 16, wherein the nucleic acid molecule is of sequence SEQ ID NO: 6.

19. The isolated nucleic acid molecule according to claim 16, wherein the nucleic acid molecule is of sequence SEQ ID NO: 7.

20. The isolated nucleic acid molecule according to claim 16, wherein the nucleic acid molecule is of sequence SEQ ID NO: 8.

21. An isolated polypeptide encoded by the nucleic acid molecule according to claim 16.

22. The isolated polypeptide according to claim 21, wherein the polypeptide is of sequence selected in the group comprising or consisting of SEQ ID NO: 2, SEQ ID NO: 3 and SEQ ID NO: 4.

23. The isolated polypeptide according to claim 21, wherein the polypeptide is of sequence SEQ ID NO: 2.

24. The isolated polypeptide according to claim 21, wherein the polypeptide is of sequence SEQ ID NO: 3.

25. The isolated polypeptide according to claim 21, wherein the polypeptide is of sequence SEQ ID NO: 4.

26. A method for the prevention and/or the treatment of an age-related disease in an individual in need thereof, comprising the administration to said individual of a therapeutically efficient amount of the isolated nucleic acid molecule according to claim 16 or of an isolated polypeptide encoded by the nucleic acid molecule.

27. The method according to claim 26, wherein the individual is a mammalian individual.

28. The method according to claim 27, wherein the individual is a human individual.

29. The method according to claim 26, wherein the age-related disease is selected in the group comprising or consisting of progeria, Werner syndrome, neurodegenerative disease, Alzheimer's disease, cancer, cardiovascular disease, obesity, type 2 diabetes, hypercholesterolemia, hypertension, ocular disorders, cataracts, glaucoma, osteoporosis, blood clotting disorders, arthritis, hearing loss and stroke.

30. The method according to claim 26, wherein the age-related disease is selected in the group comprising or consisting of Hutchinson Gilford progeria, Werner syndrome, Alzheimer's disease and cancer.

31. The method according to claim 26, wherein the age-related disease is progeria or Werner syndrome.

32. The method according to claim 26, wherein the age-related disease is cancer.

33. The method according to claim 26, wherein the isolated nucleic acid molecule or the isolated polypeptide encoded by the nucleic acid molecule is to be co-administered, or sequentially administered, with a drug suitable for preventing and/or treating an age-related disease.
